(19) United States
(12) Patent Application Publication
(10) Pub. No.: US 2024/0238131 A1
(43) Pub. Date: Jul. 18, 2024

(54) APPARATUS AND METHOD

(71) Applicant: Symbioco Ltd, London (GB)

(72) Inventors: Aaron Mathews KOSHY, London (GB); Sebastian William MACNAIR-SMITH, London (GB); Olivia Youngjoo AHN, London (GB)

(73) Assignee: Symbioco Ltd, London (GB)

(21) Appl. No.: 18/562,376

(22) PCT Filed: May 20, 2022
(86) PCT No.: PCT/GB2022/051286

§ 371 Date: Nov. 20, 2023

(30) Foreign Application Priority Data
May 20, 2021 (GB) 2107232.7

(51) Int. Cl. A61F 13/47; A61F 13/15; A61L 15/58; A61L 15/60; A61L 15/62

(52) U.S. Cl. CPC A61F 13/47; A61F 13/15211; A61F 13/15699; A61F 13/15723; A61L 15/58; A61L 15/60; A61L 15/62; A61F 2013/15235; A61F 2013/4708

(57) ABSTRACT

A method of attaching a release liner to a sanitary pad is disclosed. The method comprises: applying a pattern of adhesive, preferably water based pressure sensitive adhesive, to a sheet of release liner material; drying a sheet of release liner material comprising an adhesive pattern; overlaying a sanitary pad on the dried release liner material and adhesive pattern; and compressing the overlaid sanitary pad and dried release liner together so as to adhere the dried adhesive to the sanitary pad.

### FIELD OF INVENTION

The present invention relates to water-dispersible fluid absorbers such as a sanitary pad and methods of manufacturing water-dispersible fluid absorbers.

### BACKGROUND

Currently conventional personal hygiene products and other fluid absorbers are typically made of non-flushable materials. The proliferation of such products is a stark problem for sanitation systems worldwide; non-flushable sanitary pad which are often flushed away (1.4 million sanitary pads and 2.5 million tampons flushed every day in the UK) are known to cause blockages in sewer or cistern systems which contribute to costs of £88 million a year (Sewer Misuse 2018, Water UK).

Conventionally sanitary pads are not water dispersible. Designing water dispersible sanitary pads requires a number of technical and manufacturing challenges to be overcome. For example, existing non-water dispersible sanitary pads use several materials that are not water dispersible and that are therefore unsuitable for use in a water dispersible sanitary pad. In conventional sanitary pads these non-water dispersible materials not only impart important properties to the sanitary pads but are also important for the manufacturing of the sanitary napkin. For example, in the manufacture of conventional sanitary pads non-water dispersible constructive adhesive such as hot melt adhesive is used to provide structural cohesion to the sanitary pads during the manufacturing process.

Furthermore, in conventional sanitary pads non-water dispersible thermoplastics are also often used as adhesive for securing the sanitary napkin to an undergarment. Such non-water dispersible thermoplastics are unsuitable for water dispersible sanitary pads. The water sensitive materials of water dispersible sanitary pads present further challenges when selecting a pressure sensitive adhesive. Alternative manufacturing methods to prevent adhesives negatively impacting the properties of water sensitive materials are required.

Further challenges in the manufacture and design of water-dispersible sanitary pads are presented by the need for some materials used in water-dispersible sanitary pads to have a tensile strength low enough to comply with flushable protocols but for the materials to be sufficiently robust for use.

The present disclosure seeks to address these problems by presenting a sanitary pad which is water-dispersible and methods of manufacturing water dispersible fluid sanitary pads.

### SUMMARY

Aspects and examples of the invention are set out in the claims and aim to address at least a part of the above described technical problem, and other problems.

According to a first aspect there is provided a method of manufacturing a sanitary pad comprising an absorbent core, the method of manufacturing comprising: perforating a shape into a sheet of absorbent core material.

Perforation can permit the sheet to remain sufficiently intact for further processing, and in particular for marrying to other sheet(s), and at the same time permit removal of excess absorbent core material from around the perforated shape at the appropriate stage of processing.

The method of manufacturing preferably further comprises overlaying the sheet of absorbent core with a top sheet; and embossing a shape and/or pattern into the overlaid top sheet and sheet of absorbent core, wherein the embossed shape and/or pattern secures the top sheet to the absorbent core. Embossing can secure the top sheet to the absorbent core material sufficiently robustly for further processing, and in particular prevent slippage between the layers. The embossed shape can also enable patterning and provision of fluid distribution channels. The embossed shape may be within the perforated shape. The embossed shape may be geometrically similar to the perforated shape.

The method of manufacturing may further comprise removing excess absorbent core material from around the perforated shape to leave a top sheet and an absorbent core, wherein the absorbent core is substantially the shape of the perforated shape. This can permit removal of excess material and enable the absorbent core to be enclosed within the sanitary pad.

The method of manufacturing may further comprise overlaying the top sheet and absorbent core on a bottom sheet; and sealing the top sheet and bottom sheet together. The absorbent core may be enclosed by the top sheet and bottom sheet.

The method of manufacturing may further comprise overlaying the sheet of absorbent core on a sublayer (the sublayer preferably comprising one or more of: a polyvinyl alcohol film and a non-woven cellulosic fabric); and perforating the shape into both the sheet of absorbent core material and the sublayer.

For continuous manufacturing the absorbent core sheet, the top sheet and/or the bottom sheet are preferably continuous webs of material. For continuous manufacturing preferably a rotary machine comprising a cutting die performs the perforating step. For continuous manufacturing preferably a rotary machine comprising an embossing die performs the embossing step.

For sufficient sheet strength and sufficient ease of removal tearing preferably 90 to 99% of the perimeter of the perforated shape comprises perforation holes, further preferably wherein 95% of the perimeter of the shape comprises perforation holes.

The perforated shape may comprise an elongate shape, preferably an oval, rectangular and/or waisted shape. The embossed shape may comprise an elongate shape, preferably an oval, rectangular or waisted shape.

The embossed pattern may comprise at least one fluid distribution channel extending within the perforated shape for distribution of fluid within the perforated shape. The fluid distribution channels preferably comprise: a plurality of longitudinal channels; and a number of side channels branching out from the longitudinal channels, preferably in connection with outer adjacent longitudinal channels. The fluid distribution channels may comprise: a central longitudinal channel wherein the width of the longitudinal channel varies.

According to another aspect there is provided a method of manufacturing a sanitary pad, the method comprising: overlaying a sheet of absorbent core with a top sheet; and embossing a pattern into the overlaid top sheet and sheet of absorbent core, wherein the embossed pattern comprises at least one fluid distribution channel for distribution of fluid within the sanitary pad. The fluid distribution channel preferably comprises: a plurality of longitudinal channels; and a number of side channels branching out from the longitudinal channels, preferably in connection with outer adjacent longitudinal channels. The fluid distribution channels may comprise a central longitudinal channel wherein the width of the central longitudinal channel varies.

The method may further be as aforementioned.

According to another aspect there is provided a method of attaching a release liner to a sanitary pad comprising: applying a pattern of adhesive, preferably water based pressure sensitive adhesive, to a sheet of release liner material; drying a sheet of release liner material comprising an adhesive pattern; overlaying a sanitary pad on the dried release liner material and adhesive pattern; and compressing the overlaid sanitary pad and dried release liner together so as to adhere the dried adhesive to the sanitary pad.

This can permit application of a water-based adhesive as suitable for a water-dispersible sanitary pad, without damaging water-sensitive materials in the water-dispersible sanitary pad.

The adhesive may be applied to the release liner material in the form of a spray or in the form of a direct coating using an aperture. The adhesive may be dried by passing the release liner material through a drying tunnel.

For effective release the release liner is preferably a siliconized non-woven cellulosic fabric. For effective silicone application the release liner may comprise a layer of a water-soluble polymer, preferably a polyvinyl alcohol or a carboxymethylcellulose, optionally applied in a solution or as a melt or by lamination. The release liner may comprise a silicone layer, preferably of 0.01 to 5 gsm or around 1 gsm. The release liner may comprise a polyvinyl alcohol film, preferably of 10 to 80 μm or around 40 μm thick. The release liner may comprise a layer of a non-woven cellulosic fabric, preferably of 20 to 100 gsm or around 60 gsm weight.

The adhesive may be applied to the release liner material by steps comprising: block coating a layer of adhesive onto a surface of the release liner; applying to an exposed surface of the block coating a protective sheet, wherein the protective sheet comprises adherent portions and non-adherent portions arranged in a pattern corresponding to a desired adhesive pattern; and removing the protective sheet, wherein on removal of the protective sheet areas of the block coating in contact with the adherent portions are removed from the release liner, and areas of the block coating in contact with the non-adherent portions are left on the release liner. The non-adherent portions may be siliconized portions, and the adherent portions may be non-siliconized portions. The method may further comprise manufacturing the protective sheet by patterning, preferably by gravure coating, a silicone onto a non-siliconized sheet to form the siliconized portions.

The adhesive may be applied to the release liner material by steps comprising: doctor blade coating (and/or reverse roll coating) a water-based adhesive onto a non-water-dispersible liner; drying the adhesive; and transferring the dried adhesive to the release liner.

The adhesive may be applied to the release liner material by steps comprising: patterning the release liner to comprise adherent portions and non-adherent portions arranged in a pattern corresponding to a desired adhesive pattern; and block coating a layer of adhesive onto a surface of the release liner; such that in use, on removal of the release liner areas of the block coating in contact with the non-adherent portions are removed from the release liner, and areas of the block coating in contact with the adherent portions are left on the release liner.

The adhesive is preferably a water-soluble or water-dispersible adhesive. The release liner is preferably a water-soluble or water-dispersible release liner.

The method may further be as aforementioned.

According to another aspect there is provided a method of patterning an adhesive onto a release liner, wherein the method comprises: block coating a layer of adhesive onto a surface of the release liner; applying to an exposed surface of the block coating a protective sheet, wherein the protective sheet comprises adherent portions and non-adherent portions arranged in a pattern corresponding to a desired adhesive pattern; and removing the protective sheet, wherein on removal of the protective sheet areas of the block coating in contact with the adherent portions are removed from the release liner, and areas of the block coating in contact with the non-adherent portions are left on the release liner. The adhesive may be a water-soluble or water-dispersible adhesive. The release liner is preferably a water-soluble or water-dispersible release liner. The non-adherent portions may be siliconized portions, and the adherent portions may be non-siliconized portions. The method may further comprise manufacturing the protective sheet by patterning, preferably by gravure coating, a silicone onto a non-siliconized sheet to form the siliconized portions.

The method may further be as aforementioned.

According to another aspect there is provided a method of patterning a water-dispersible release liner, wherein the method comprises: doctor blade coating and/or reverse roll coating a water-based adhesive onto a non-water-dispersible liner; drying the adhesive; and transferring the dried adhesive to the release liner.

Transferring the adhesive to the release liner may comprise contacting the patterned adhesive to the release liner. The doctor blade (or a profile of a metering roll of the reverse roll coating) may form a number of gaps to coat the water-based adhesive onto the non-water-dispersible liner in a strip pattern. The water-based adhesive is preferably a water-soluble or water-dispersible adhesive. The non-water-dispersible liner may be a clay paper or a glassine paper.

The method may further be as aforementioned.

According to another aspect there is provided a method of patterning a release liner, wherein the method comprises: patterning the release liner to comprise adherent portions and non-adherent portions arranged in a pattern corresponding to a desired adhesive pattern; and block coating a layer of adhesive onto a surface of the release liner; such that in use, on removal of the release liner areas of the block coating in contact with the non-adherent portions are removed from the release liner, and areas of the block coating in contact with the adherent portions are left on the release liner.

The adhesive is preferably a water-soluble or water-dispersible adhesive. The release liner is preferably a water-soluble or water-dispersible release liner. The non-adherent portions may be siliconized portions, and the adherent portions may be non-siliconized portions. The method may comprise patterning the release liner by patterning a silicone onto a non-siliconized sheet to form the siliconized portions, preferably by spraying or gravure coating.

The method may further be as aforementioned.

According to another aspect there is provided a method of manufacturing a bottom layer of a sanitary pad, the method comprising: priming a non-woven cellulosic fabric for application of an adhesive by applying an aqueous solution of polyvinyl alcohol or carboxymethylcellulose; and drying the primed non-woven cellulosic fabric. Priming and drying can fortify the non-woven cellulosic fabric and improve adhesive performance.

For effective priming the aqueous solution may be about 0.1-5% by weight polyvinyl alcohol or carboxymethylcellulose in water, preferably around 2% by weight polyvinyl alcohol in water. The aqueous solution may comprise a high hydrolysis polyvinyl alcohol, preferably with a degree of hydrolysis of over 80 mol %. The aqueous solution of polyvinyl alcohol or carboxymethylcellulose may be applied by spraying, gravure or slot die.

The primed non-woven cellulosic fabric may comprise a coating of polyvinyl alcohol or carboxymethylcellulose that is less than 1 μm thick and/or less than 1 gsm, preferably 1-1000 milligram per square meter (mgsm). The coating thickness may be in the range of 1-500 mgsm or 500-1000 mgsm or 1-250 mgsm or 250-500 mgsm or 500-750 mgsm. To afford sufficient priming the coating thickness may be at least 1 mgsm or at least 10 mgsm or at least 50 mgsm or at least 100 mgsm or at least 250 mgsm. To avoid reducing the effectiveness of the priming the coating thickness may be less than 1000 mgsm or less than 750 mgsm or less than 500 mgsm or less than 250 mgsm. The non-woven cellulosic fabric may have a weight of 20 to 100 gsm or around 60 gsm weight.

The method may further comprise contacting a pressure sensitive adhesive to the primed non-woven cellulosic fabric. The method may further comprise extruding or laminating a layer of polyvinyl alcohol onto the opposite face of the non-woven cellulosic fabric.

The primer may form a polyvinyl alcohol film on the non-woven cellulosic fabric, preferably less than 1 μm thick. The pressure sensitive adhesive is preferably a water-soluble pressure sensitive adhesive, and may be included at around 8-10 gsm.

The method may further be as aforementioned.

According to another aspect there is provided a method of bonding a fibrous material and a polyvinyl alcohol film, comprising: applying a polyvinyl alcohol solution to a surface of the polyvinyl alcohol film and/or a surface of the fibrous material; and contacting the surface of the polyvinyl alcohol with the surface of the fibrous material such that the applied polyvinyl alcohol solution is positioned between the polyvinyl alcohol film and the fibrous material.

The method may further comprise compressing the contacted polyvinyl alcohol film and fibrous material together, preferably in the absence of heating. The polyvinyl alcohol solution may be one or more of:
a solution of high hydrolysis polyvinyl alcohol, preferably over 80 mol % hydrolysis;
1-15% by weight polyvinyl alcohol in solvent, preferably around 5% by weight polyvinyl alcohol in solvent;
an aqueous solution; and
applied at less than 5 gsm, preferably around 1 gsm.

The fibrous material and the polyvinyl alcohol film may at least partially form a sublayer of a sanitary pad. Preferably the fibrous material and the polyvinyl alcohol film at least partially form a bottom sheet of a sanitary pad.

The polyvinyl alcohol solution may be one or more of:
a solution of high hydrolysis polyvinyl alcohol, preferably over 80 mol % hydrolysis;
10-20% by weight polyvinyl alcohol in solvent, preferably around 15% by weight polyvinyl alcohol in solvent;
an aqueous solution; and
applied to form a film of more than 1 gsm (dry), preferably more than 3 gsm (dry); and/or less than 15 gsm (dry), preferably less than 12 gsm (dry).

The fibrous material may at least partially form an absorbent core of a sanitary pad. The fibrous material may at least partially form a top sheet of a sanitary pad. The polyvinyl alcohol film may at least partially form a bottom sheet of the sanitary pad.

The fibrous material may be a non-woven fabric or a loose fibre material.

The method may further be as aforementioned.

According to another aspect there is provided a sanitary pad comprising a reinforced bottom layer wherein the bottom layer has been manufactured using the method as aforementioned.

According to another aspect there is provided a sanitary pad comprising a reinforced bottom layer, with the bottom layer comprising: a layer of a non-woven cellulosic fabric with a polyvinyl alcohol film on a first surface; and a polyvinyl alcohol coating on the other surface. The polyvinyl alcohol film on the first surface may be around 10-80 μm thick. The polyvinyl alcohol coating on the other surface may be less than 1 μm thick.

According to another aspect there is provided a method of assembling a sanitary pad and release liner, the method comprising overlaying a sanitary pad onto an adhesive pattern arranged on a release liner.

By arranging the adhesive pattern on the release liner the adhesive pattern can be prepared without damaging potentially water-sensitive materials of the sanitary pad. This can be particularly useful in the context of manufacturing a water-dispersible sanitary pad. Preferably the sanitary pad is overlaid in a folded configuration in which tabs of the sanitary pad are folded over onto a main body of the sanitary pad. This can permit particularly efficient manufacturing and packaging of the sanitary pad.

For efficient manufacturing and packaging the method may further comprise cutting from the release liner a shape complementary to the shape of the sanitary pad in the unfolded configuration, wherein the cut release liner shape comprises wing sections extending from a central section of the cut release liner shape, with an adhesive pattern arranged on the wing sections; folding the wing sections of the cut release liner onto the folded tabs of the sanitary pad; and compressing the folded release liner so as to adhere the adhesive pattern and release liner to the wings of the sanitary pad. The method may further comprise folding a first section of the release liner and sanitary pad to overlay the wing sections; and folding a second section of the release liner and sanitary pad to overlay the first section. The adhesive is preferably a water-soluble or water-dispersible adhesive. The release liner is preferably a water-soluble or water-dispersible release liner.

The method may further be as aforementioned.

According to another aspect there is provided a sanitary pad produced by the manufacturing methods as aforementioned. It will be appreciated that the aforementioned methods may be combined in any suitable combination.

According to another aspect there is provided an acquisition distribution layer for a sanitary pad comprising polyvinyl alcohol fibres, preferably long polyvinyl alcohol fibres. This can permit provision of a biodegradable sanitary pad and/or a flushable sanitary pad.

The long polyvinyl alcohol fibres may be at least 1 mm long, at least 3 mm long, at least 10 mm long, at least 20 mm long, at least 25 mm long, at least 30 mm long, at least 40 mm long, at least 50 mm long, or at least 60 mm long. The polyvinyl fibres may be hollow and tubular in shape

The acquisition distribution may be formed partially or substantially of polyvinyl alcohol fibres. Preferably the polyvinyl alcohol fibres are arranged in parallel. Preferably the polyvinyl alcohol fibres are arranged to form a non-woven fabric. The acquisition distribution may further include wood pulp and/or or viscose fibres.

According to another aspect there is provided a sanitary pad comprising an acquisition distribution layer as aforementioned. The sanitary pad may be as aforementioned.

According to another aspect there is provided a water dispersible sanitary pad comprising: a top layer of a non-woven cellulosic fabric, preferably having 20-100 gsm or around 60 gsm weight;

an absorbent layer comprising:
fiberised cellulose wood pulp, preferably having 70-300 gsm or around 250 gsm weight;
a super absorbent polymer, preferably a cellulose gum, further preferably 0.1-5 g or around 1 g of super absorbent polymer per sanitary pad; and
optionally a first carrier layer of a non-woven cellulosic fabric, preferably having 5-100 gsm or around 60 gsm weight;
   an optional sublayer comprising:
a polyvinyl alcohol film, preferably 10-80 μm or around 20 μm thick or with a dry weight of more than 1 gsm, preferably more than 3 gsm; and/or less than 15 gsm, preferably less than 12 gsm; and
optionally a second carrier layer of a non-woven cellulosic fabric, preferably having 5-100 or 20-100 gsm or around 60 gsm weight;
   a bottom sheet comprising:
a polyvinyl alcohol film, preferably 10-80 μm or around 30 μm thick; and
a layer of a non-woven cellulosic fabric, preferably having 5-100 or 20-100 gsm or around 60 gsm weight;
optionally a polyvinyl alcohol film, preferably less than 1 μm thick; and
   a water-soluble pressure sensitive adhesive, preferably included at around 8-10 gsm.

This particular combination of materials is found to provide a particularly effective balance between the requirements of water-dispersibility against sufficient robustness for use and suitability for high-speed continuous manufacturing.

The water dispersible sanitary pad may further comprise a release liner comprising:
a silicone layer, preferably having 0.1-5 or 1-4 or around 1 gsm;
a polyvinyl alcohol film, preferably more than 10-μm or 10-100 μm or around 40 μm thick; and
a layer of a non-woven cellulosic fabric, preferably having 5-100 or 20-100 or around 60 gsm weight.

The sanitary pad may be as aforementioned.

According to another aspect there is provided a sanitary pad comprising: an absorbent core; a bottom layer comprising a non-woven fabric and a polyvinyl alcohol film; and a sublayer arranged between the absorbent core and the bottom layer, the sublayer comprising a polyvinyl alcohol film and optionally a non-woven fabric.

The sublayer can provide a barrier to moisture from the absorbent core, in particular with the polyvinyl alcohol film preventing liquid from passing, and the non-woven fabric permitting vapour to be absorbed.

Preferably the polyvinyl alcohol film of the sublayer is provided adjacent to the absorbent core. The polyvinyl alcohol film of the sublayer may be between 10 and 80 μm thick, preferably between 15 and 25 μm thick, and further preferably around 20 μm thick. The non-woven fabric of the sublayer and/or the non-woven fabric of the bottom layer may be formed of a non-woven wet laid cellulosic fibre material, preferably having 5-100 or 20-100 gsm or around 60 gsm weight. The polyvinyl alcohol film of the bottom layer may be between 10 and 80 μm thick, preferably between 30 and 50 μm thick, and further preferably around 20 or 30 μm thick.

The sanitary pad may comprise two or more sublayers arranged between the absorbent core and the bottom layer, preferably with each of the two or more sublayers comprising a polyvinyl alcohol film and a non-woven fabric. Optionally the cumulative thickness of the polyvinyl alcohol films of the two or more sublayers may be between 10 and 80 μm thick. Optionally the cumulative mass of the non-woven fabric of the two or more sublayers may be between 5-100 or 20-100 gsm.

The sublayer may be a bonding sublayer formed by application of a polyvinyl alcohol solution to the polyvinyl alcohol film of the bottom layer. The polyvinyl alcohol film of the sublayer may have a dry weight of more than 1 gsm, preferably more than 3 gsm; and/or less than 15 gsm, preferably less than 12 gsm. The polyvinyl alcohol solution may be in the range of 10-20% by weight polyvinyl alcohol in water, preferably around 15%. The polyvinyl alcohol solution may be a solution of high hydrolysis polyvinyl alcohol, preferably over 80 mol % hydrolysis.

The sanitary pad may be as aforementioned.

It will be appreciated that the aforementioned features of a sanitary pad may be combined in any suitable combination.

As used herein, the terms ‘water-dispersible’, ‘water-soluble’ and ‘biodegradable’ preferably mean compatible with the UK SNAP protocol for testing disintegration in drainline and/or compatible with the UK Fine-to-Flush protocol for testing disintegration in drainline and/or compatible with the INDA EDANA “Guidelines for Assessing the Flushability of Disposable Nonwoven Products” and/or compatible with the IWSFG Slosh Box Disintegration Test and/or compatible with one of the OECD 301 tests.

As used herein, the term ‘sanitary pad’ preferably means a pad suitable for attachment to an undergarment and suitable for absorbing a flow of blood from the vagina. A sanitary pad may also be referred to as a sanitary napkin, sanitary towel or menstrual pad. As used herein the term ‘sanitary pad’ preferably includes maternity pads, panty liners, and night pads. The term ‘sanitary pad’ may include incontinence pads and/or hemorrhoid pads.

As used herein gsm means grams per square metre and mgsm means milligrams per square metre.

### BRIEF DESCRIPTION OF DRAWINGS

Some practical implementations will now be described, by way of example only, with reference to the accompanying drawings in which:

FIG. 1 shows a schematic of layers of a sanitary pad;

FIG. 2 shows a view of the underside of a sanitary pad;

FIG. 3 shows a flow diagram of a part of a process for manufacturing a sanitary pad;

FIG. 4 shows a schematic of an assembly line for a sanitary pad;

FIG. 5 shows a top view of an embossed pattern and shape;

FIG. 6 shows a flow diagram of a method of manufacturing a bottom layer of a sanitary pad;

FIG. 7 shows a flow diagram of part of a method of manufacturing a sanitary pad;

FIG. 8 shows a schematic of an assembly line for a sanitary pad;

FIG. 9 shows a flow diagram of part of a method of manufacturing and packaging a sanitary pad;

FIGS. 10a, 10b, 10c, 10d, and 10e show a sanitary pad at various stages of the assembly illustrated in FIG. 9;

FIG. 11 shows a schematic of layers of a sanitary pad;

FIG. 12 shows a schematic plan view of a protective sheet for making an adhesive pattern;

FIG. 13 shows a schematic cross sectional view of a release liner with an adhesive block coating and a protective sheet;

FIG. 14 shows a method of applying an adhesive pattern to a release liner;

FIGS. 15a, 15b, 15c and 15d show schematics of variants of a bottom sheet;

FIG. 16 shows another method of applying an adhesive pattern to a release liner; and

FIG. 17 shows apparatus for carrying out the method shown in FIG. 16.

### SPECIFIC DESCRIPTION

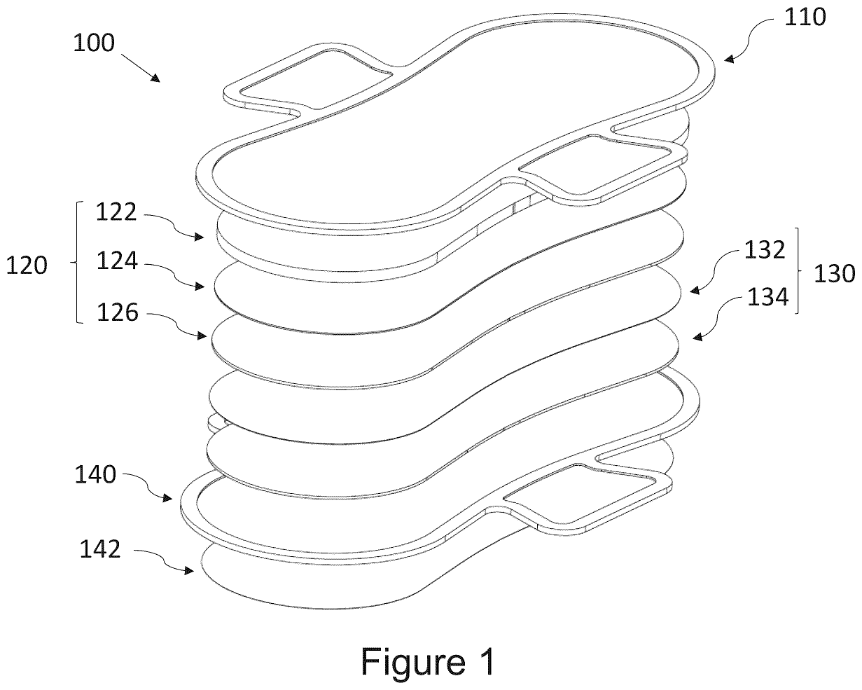

FIG. 1 shows an example of a sanitary pad 100. An absorbent layer 120 is enclosed between a top layer 110 and a bottom layer 140.

The sanitary pad 100 is able to absorb bodily fluids and subsequent to use can disperse well in water at ambient temperatures. It is recognised that finding a combination of materials and bonding them appropriately, such that the requirement to absorb fluid whilst remaining stable and also the requirement to disperse are fulfilled, is a challenge. It is also recognised that such requirements, materials and novel material combinations present new manufacturing challenges.

The top layer 110 of the sanitary pad 100 has high fluid permeability to ensure that body fluid is able to pass quickly through the top layer 110 to the interior of the sanitary pad 100. It is desirable for body fluid to be transferred rapidly away from the top layer 110; therefore a material with a high wicking value is desirable for the top layer 110. The top layer 110 is typically a cellulosic non-woven fabric. The cellulosic fibres may for example be natural cellulose fibres or manufactured cellulose fibres, such as rayon fibres including viscose rayon. Other suitably dispersible top layers may be used. A suitable top layer may for example have a density of 25-75 grams per square metre (gsm), 30-65 gsm, or approximately 60 gsm.

In an example short-cut Viloft viscose rayon fibres are processed into a non-woven fabric through a wet-laid spunlace process. In another example a wet-laid semi-hydrophobic top layer is substantially of wood pulp and hydrophobic cellulosic fibre (such as Olea hydrophobic viscose rayon fibres from Kelheim Fibres). A wet-laid material is observed to have superior water-dispersibility. A semi-hydrophobic material can provide a favourable dry feel as the moisture is wicked to the absorptive core rather than remaining on the surface of the sanitary pad.

Advantageously the top layer 110 is a wet-laid spunlace (WLS). Spunlace (also referred to as hydroentanglement) is formed by a mechanical process that requires no binder. Dispersibility can be higher with WLS than with air-laid/dry-laid fabric as no binder is used. WLS material may have a relatively low wet strength, which may be acceptable for use in a sanitary pad or similar where the material goes from dry to wet and is not subject to excessive shear force during use (until exposed to the hydraulic action in a toilet). To remain highly dispersible, the web of the WLS may be a parallel-laid (also referred to as parallel lapped) material. Compared to a cross-laid material a parallel-laid material can also provide higher tensile strength, lower elongation and lower tear strength.

The sanitary pad 100 shown in FIG. 1 has a top layer 110 comprising a 100% cellulosic non-woven spun lace material having a density of 60 gsm such as those available under the trade name Jacob Holm™ Softflush.

Examples of properties of a 100% cellulosic non-woven spun lace material having a density of 60 gsm that is suitable as a top sheet are:

| | Orientation | | | | |
|---|---|---|---|---|---|
| | (Machine Direction | | | | |
| | MD, or Cross | | | | Measurement |
| Characteristic | Direction CD) | Units | Target | Range | method |
| Basis Weight | | g/m² | 60 | 52-68 | WSP 130.1 |
| Thickness | | mm | 0.65 | 0.55-0.75 | WSP 120.6 |
| Tensile Strength | MD | N/5 cm | 6.6 | 5.1-8.1 | WSP 110.4 |
| | CD | | 4.5 | 3.0-6.0 | |
| Elongation at break | MD | N/5 cm | 18 | 8-28 | WSP 110.4 |
| | CD | | 32 | 17-48 | |
| Absorption capacity | | % | 800 | 600-1000 | WSP 10.0 |

The absorbent core 120 of sanitary pad 100 is formed of a layer of a fiberised cellulose wood pulp layer 122 and a first carrier layer 126 of wet-laid non-woven fibres. In an alternative the wood pulp is replaced with a high gsm (e.g. 70-300 gsm) water dispersible non-woven material, or a mixture of both is used. Preferably a super absorbent polymer 124 is incorporated in the absorbent core 120 to enhance the absorbent properties. The absorbent core 120 of the sanitary pad 100 absorbs and retains fluids thereby preventing fluid from passing through the sanitary pad 100. To enable biodegradability the fibres used in the absorbent core 120 are preferably cellulosic.

The cellulosic fibres may for example be natural cellulose fibres such as wood pulp fibres, or manufactured cellulose fibres, such as rayon fibres including viscose rayon.

The absorbent core 120 may include a blend of different fibres, for example a blend of both wood pulp fibres and viscose rayon fibres. Examples of suitable cellulosic fibres include those available under the trade name Danufil, Galaxy or Viloft from Kelheim Fibres or others.

The super absorbent polymer 124 may be a partially cross-linked cellulose-based material such as a sodium carboxymethylcellulose. Cellulose-based superabsorbents are biodegradable and food safe, unlike other super absorbent polymers conventionally used (such as polyacrylamides or polyacrylates). The super absorbent polymer 124 may be an alginate material. The super absorbent polymer 124 may be a cellulose-based material such as the cellulose gum available under the trade name of Aquasorb® from Ashland.

If a super absorbent polymer 124 is included in the absorbent core 120, then the absorbent core 120 may be formed of a layer of super absorbent polymer 124 sandwiched between two layers of fibres, or a layer of super absorbent polymer 124 sandwiched between a layer of fibres and a bottom layer 140. Alternatively, the absorbent layer 120 may comprise super absorbent polymer 124, preferably in powder form, homogeneously distributed between the two layers of fibres (122, 126) or within one or more of the two fibre layers.

The absorbent core 120 of sanitary pad 100 is formed of:
a layer of fiberised cellulose wood pulp 122 e.g. having a density of 250 gsm, or alternatively a high gsm (e.g. 70-300 gsm) water dispersible non-woven;
a super absorbent polymer 124 (SAP), such as a cellulose gum (carboxymethylcellulose, CMC), e.g. as available under the tradename Aquasorb A500 from Ashland; and
a first carrier layer 126 of wet-laid non-woven fibres, e.g. having a density of 60 gsm for example a wet-laid cellulosic non-woven available under the tradename Hydraspun™ Royal from Suominen

The super absorbent polymer is homogenously distributed in powder form in a layer between the fiberised cellulose wood pulp and the first carrier layer.

Examples of properties of a wet-laid non-woven that is suitable as a first carrier layer are:

| | Orientation | | | |
|---|---|---|---|---|
| | (Machine Direction | | | |
| | MD, or Cross | | Typical | Measurement |
| Characteristic | Direction CD) | Units | Value | method |
| Basis Weight | | g/m² | 60 | EDANA 40.3-89 |
| Thickness | | mm | 0.6 | Tappi T-311 |
| Dry Tensile Strength | MD | N/m | 600 | EDANA 20.2-89 |
| | CD | | 300 | |
| Elongation at break | MD | N/m | 135 | EDANA 20.2-89 |
| | CD | | 90 | |
| Absorption capacity | | % by weight | 750 | EDANA 20.2-89 |

An example of a cellulose gum that is suitable as a super absorbent polymer is a 99.5% purity (by weight) sodium carboxymethylcellulose with following properties:

| | | Typical | Measurement |
|---|---|---|---|
| Characteristic | Units | Value | method |
| Loss on drying | % by weight | 0-10 | MA 304.1500 |
| pH | | 6.5-8.5 | MA 304.1004 |
| Tea bag test 10 | g/g | 30 | MA 304.1402 |
| minutes | | | |
| Sodium content | % by weight | 0-12.4 | D 304.208 |
| Sodium glycolates | % by weight | 0-0.4 | MA 304.1502 |

An optional sublayer 130 of the sanitary pad 100 forms a fluid impermeable layer and comprises a polyvinyl alcohol (PVA, also referred to as PVOH) layer 132, and a second carrier layer 134. The sublayer 130 prevents fluid and moisture that may leak from the absorbent core 100 from reaching a bottom surface of the sanitary pad 100. In particular the sublayer 130 provides a barrier to moisture from the absorbent core 120 thereby protecting the integrity of a bottom sheet 140 of the sanitary pad 100 described below. The inclusion of a sublayer 130 is optional but has been found to improve the performance of the sanitary pad 100 by preventing fluid or moisture from escaping the absorbent core 120 and adversely affecting the bottom sheet 140 of the sanitary pad 100.

The PVA film 132 of the sublayer 130 is a water-soluble PVA or a water-dispersible PVA, such as those available under the trade name Solublon or Hydropol. The melting point of a thermoplastic PVA film is typically 140° C.-200° C. A 25 μm thick film of a suitable PVA such as Hydropol™ 33200P from Aquapak™ has a solubility at 60° ° C. in tap water of <60 seconds as measured under test method AQU QALAB SOP 4. In an example the PVA layer 130 provided in sanitary pad 100 is 20 μm thick; this thickness has been found to be effective at protecting the bottom sheet 140 from moisture whilst not significantly adversely affecting the other properties of the sanitary pad 100 such as weight or thickness. Other thicknesses of PVA film have found to be effective, for example films of thickness between 10 and 30 μm.

The second carrier layer 134 is formed from a non-woven fabric having similar properties to the non-woven fabric of the first carrier layer 126. The second carrier layer 134 of the sanitary pad 100 shown in FIG. 1 is a wet-laid cellulosic non-woven fabric such as those available under the tradename Hydraspun™ Royal from Suominen, having a density of 60 gsm and a thickness of 0.6 mm. The second carrier layer 134 provides additional protection to the bottom sheet 140 through absorption of moisture and improves the structural strength of the sanitary pad 100. The terminal layer of the sanitary pad 100 is the bottom sheet 140. The bottom sheet 140 comprises a wet-laid cellulosic non-woven fabric such as those used in the first and second layers. However, in the case of the bottom layer 140 the non-woven fabric is reinforced by a PVA layer. In one example the bottom sheet 140 of sanitary pad 100 has a non-woven fabric layer with a PVA layer extruded onto the surface of the non-woven fabric. In another example a PVA layer is thermally laminated to the non-woven fabric. Extrusion coating can permit the PVA layer to be better integrated with the non-woven fibres but can be more complex to implement.

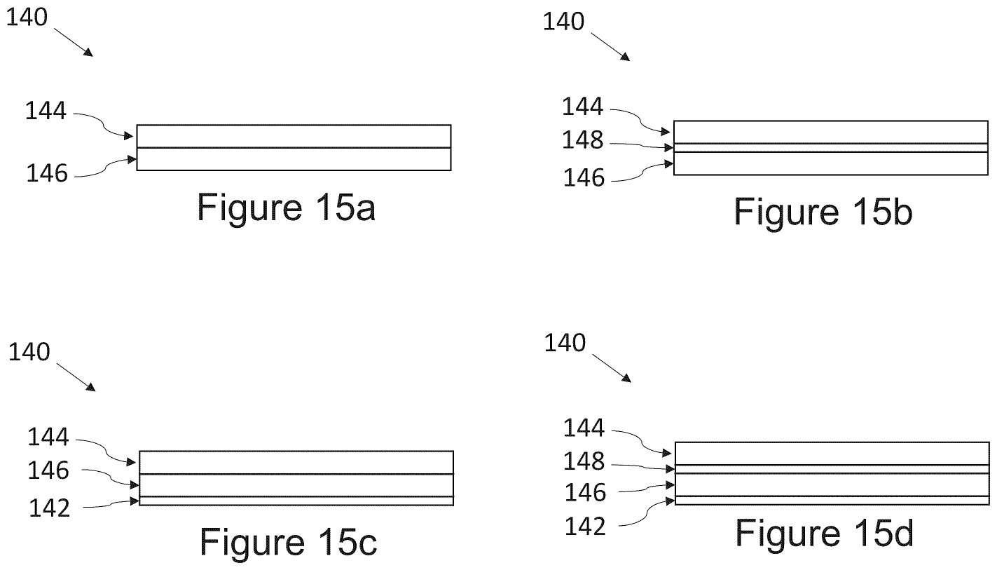

Alternatively, a PVA (PVOH) solution may be used between the non-woven fabric layer and a PVA layer to bind a PVA film to the non-woven fabric of the bottom sheet 140. This variant is illustrated in FIG. 15. In more detail, a PVA solution is applied to the non-woven for example by spray, gravure, slot die or another method of application. A PVA film is positioned over the sprayed surface. The PVA film, non-woven fabric and intermediate PVA solution on the surface of the non-woven fabric are then compressed together (preferably before the sprayed surface has dried), for example using a rotary press. Due to the inclusion of the binder layer of PVA solution the PVA film binds readily to the primed non-woven fabric and a bond between the PVA film and the non-woven fabric can be achieved without heating, unlike with thermal lamination.

High hydrolysis PVA solutions have been found to be particularly effective at binding the PVA film to the non-woven fabric, for example PVA solutions with a degree of hydrolysis over 70 mol % or over 80 mol % and preferably with a degree of hydrolysis below 99 mol % or below 95 mol %, e.g. 87-89 mol %. High hydrolysis PVA is preferably over 80 mol % hydrolysed, i.e. residual vinyl acetate units make up less than 20 mol %. High hydrolysis PVA can be less sensitive to cold water and perform well as a binder. The PVA solution may be applied at e.g. 1 gsm, and generally below 5 gsm. In an example the PVA solution is 5% by weight PVA in water; the primer is typically in the range of about 0.1-15% by weight polyvinyl alcohol in water.

The PVA solution is thought to coat the fibres of the non-woven fabric, particularly those fibres on the surface of the non-woven fabric. The coated fibres provide improved binding between the PVA film and the non-woven fabric. The improved binding means that compression “cold lamination” can be used to construct the bottom sheet or sublayer as an alternative to thermal lamination or extrusion. Thermal lamination or extrusion can however still be used as an alternative to compression.

Alternatively, or in addition, to application of the PVA solution to the surface of the non-woven fabric the PVA solution may be applied to the surface of the PVA film. The PVA solution may be applied in different patterns across the surface of the non-woven fabric and/or PVA film for example it may be applied uniformly or it may be applied in a discontinuous pattern.

A PVA solution may also be used in a similar manner to improve binding between the PVA film 132 and second carrier layer 134 of the sublayer 130 or indeed to improve the binding between any non-woven fabric and a PVA film.

The PVA film, whether by hot or cold lamination or by extrusion, has similar properties to the PVA film of the sublayer 130. The PVA film is a water-soluble PVA and/or a water dispersible PVA. The PVA layer is for example 20-50 μm thick. This thickness has been found to provide sufficient structural reinforcement to the bottom sheet 140 whilst not adversely affecting the water-dispersibility of the sanitary pad 100. Other thicknesses of the PVA layer have also been found to be effective, for example a PVA layer between 10 and 80 μm, preferably between 20 and 60 μm. The bottom sheet is arranged such that the PVA layer faces the absorbent layer.

The non-woven fabric of the bottom layer may be prepared for accepting an adhesive layer by applying a primer layer or coating 142 to the exposed surface of the non-woven fabric. A suitable primer comprises PVA or CMC dissolved in water, and the primer may be applied by spraying the primer onto the non-woven fabric or by other methods. The primer treats one surface of the fabric, whilst the other surface has a PVA film arranged on it as described above, on the opposite side of the fabric. The non-woven fabric is then dried to evaporate the water. This process is described in more detail below.

FIGS. 15a, 15b, 15c and 15d are schematics of some variants of the bottom sheet 140 as described above. FIG. 15a shows a bottom sheet 140 with a PVA film 144 thermally laminated or extruded onto a non-woven fabric 146. FIG. 15b shows a bottom sheet 140 with a PVA binder layer 148 applied from a PVA solution between a PVA film 144 and a non-woven fabric 146. FIG. 15c shows a bottom sheet 140 with a PVA primer layer 142 applied to the exposed surface of the non-woven fabric 146. FIG. 15d shows a bottom sheet 140 with a PVA binder layer 148 between a PVA film 144 and a non-woven fabric 146 as well as a PVA primer layer 142 applied to the exposed surface of the non-woven fabric 146.

Sanitary pads are typically provided with a pattern of adhesive to secure the sanitary pad to an undergarment in use and to secure a release liner to the bottom sheet prior to use. The reinforcement of the non-woven fabric of the bottom layer 140 with an extruded PVA film advantageously improves the adherence of the adhesive to the non-woven fabric and also improves the structural integrity of the non-woven fabric. This improvement in structural integrity is of particular importance during use of the sanitary pad 100 when it is removed from the undergarment. In particular it is desirable to have a non-woven material that has a tensile strength greater than the peel strength of the adhesive so that when the sanitary pad is removed from the undergarment the adhesive remains on the non-woven rather than the undergarment. Furthermore, a non-woven material with a low tensile strength particularly a tensile strength that is lower than the peel strength of the adhesive can become damaged or torn during removal. Tearing of the non-woven during removal might result in the adhesive, and a torn section of the non-woven, remaining on the undergarment. The reinforcement of the non-woven material advantageously improves its tensile strength.

The sanitary pad 100 has a layered structure with the top sheet 110 forming the top layer. Adjacent the top layer 110 is the absorbent core 120. The layers of the absorbent core are arranged so that the wood pulp layer 122 is adjacent the top layer 110, the distributed SAP layer 124 is adjacent the wood pulp layer 122 and adjacent the first carrier layer 126. The sublayer 130 is optionally provided adjacent to the absorbent core 120—the absorbent core 120 is provided between the top sheet 110 and the bottom sheet 140. The layers of the sublayer 130 are arranged such that the PVA layer 132 is adjacent the absorbent core 120, specifically the first carrier layer 126 of the absorbent core 120, and the second carrier layer 134 is adjacent the PVA layer 132 of the bottom sheet. The sanitary pad 100 is terminated by the bottom sheet 140. The bottom sheet 140 is arranged to have the PVA film adjacent to the optional sublayer; the PVA primer treated face is on the opposing side of the bottom sheet 140, adjacent to the garment during use.

The structure and materials of an example sanitary pad are summarised in the table below. It will be appreciated that these materials and dimensions may be varied.

| | | | Approximate |
|---|---|---|---|
| Layer | Material | Thickness | Density/Weight |
| Top Layer | Non-woven cellulosic | Typically in the range of | Typically in the range of |
| | wet-laid spunlace | 0.15-1.15 mm, e.g. 0.55- | 5-80 gsm, e.g. 60 gsm |
| | material | 0.75 mm | |
| Absorbent | Fiberised cellulose | Typically in the range of | Typically in the range of |
| core | word pulp | 0.5-10 mm, e.g. 2-10 mm | 100-500 gsm, e.g. 250 gsm |
| | Super absorbent | | Typically in the range of |
| | polymer | | 0.5-3 g per sanitary pad, |
| | | | e.g. 1 g per sanitary pad |
| | Non-woven wet-laid | Typically in the range of | Typically in the range of |
| | cellulosic fibre | 0.15-1.15 mm, e.g. 0.6 mm | 5-80 gsm, e.g. 60 gsm |
| Sub layer | PVA | Typically in the range of | Typically in the range of |
| | | 5-60 μm, e.g. 20 μm film | 5-80 gsm, e.g. 25 gsm |
| | Non-woven wet-laid | Typically in the range of | Typically in the range of |
| | cellulosic fibre | 0.15-1.15 mm, e.g. 0.6 mm | 5-80 gsm, e.g. 60 gsm |
| Bottom | PVA | Typically in the range of | Typically in the range of |
| Sheet | | 5-60 μm, e.g. 30 μm film | 5-80 gsm, e.g. 40 gsm |
| | Non-woven wet-laid | Typically in the range of | Typically in the range of |
| | cellulosic fibre | 0.15-1.15 mm, e.g. 0.6 mm | 5-80 gsm, e.g. 60 gsm |

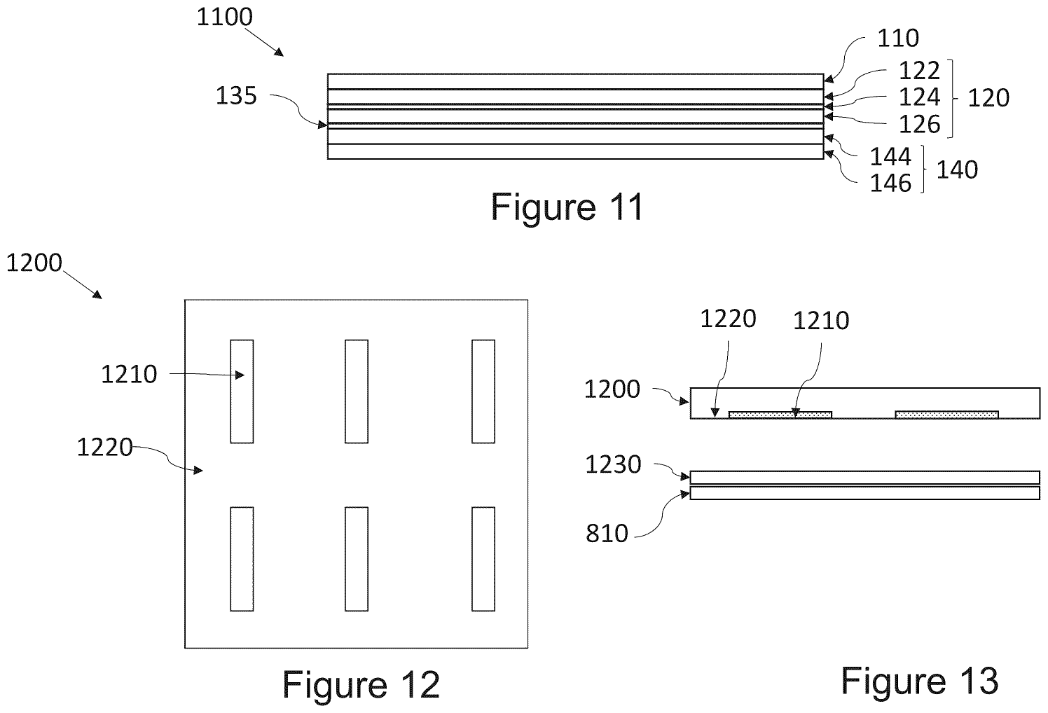

An alternative sanitary pad 1100 is shown in FIG. 11. The sanitary pad 1100 has a similar composition to sanitary pad 100 described above, however the optional sublayer 130 is omitted and bonding sublayer 135 is included instead. The PVA layer 144 of the bottom sheet 140 is bonded to the first carrier layer 126 of the absorbent core 120 by way of the bonding sublayer 135. The bonding sublayer 135 is formed by applying a PVA solution onto the surface of the PVA layer 132 of the bottom sheet 140. After application of the PVA solution to the surface of a PVA film a tacky or sticky bonding sublayer 135 forms which can be very effective for bonding to another layer, here the absorbent core and the top sheet 110. As the bonding sublayer 135 is sticky after application (when ‘wet’), it can serve to fix the bottom sheet to the absorbent layer during assembly and manufacturing of the sanitary pad; it can even be used as a constructive adhesive to bond the bottom sheet to the absorbent layer during use. The bonding sublayer 135 can also assist in forming a strong and humid resistant seal around the perimeter of the pad, for example if the perimeter is bonded with thermo embossing or ultrasonic bonding. Use of the bonding sublayer 135 to bind the bottom sheet 140 and the absorbent core 120 can improve the leak resistance of the sanitary pad.

The PVA solution is preferably applied by slot die application, but it may be applied by other methods instead (e.g. spray or gravure application). The bottom sheet, the bonding sublayer 135 and the absorbent layer can be compressed together for example using a rotary press.

High hydrolysis PVA solutions have been found to be particularly effective at binding the PVA film to the non-woven fabric, for example PVA solutions with a degree of hydrolysis over 70 mol % or over 80 mol % and preferably with a degree of hydrolysis below 99 mol % or below 95 mol %, e.g. 87-89 mol %. The PVA solution may be about 10-20% by weight polyvinyl alcohol in water, preferably around 15% by weight polyvinyl alcohol in water. The PVA solution may be applied to form a bonding sublayer 135 of e.g. 1 gsm (dry) or more, preferably in the range of 3-12 gsm (dry) and generally below 15 gsm (dry).

The structure and materials of an example sanitary pad are summarised in the table below. It will be appreciated that these materials and dimensions may be varied.

| | | | Approximate |
|---|---|---|---|
| Layer | Material | Thickness | Density/Weight |
| Top Layer | Non-woven cellulosic | Typically in the range of | Typically in the range of |
| | wet-laid spunlace | 0.15-1.15 mm, e.g. 0.55- | 5-80 gsm, e.g. 60 gsm |
| | material | 0.75 mm | |
| Absorbent | Fiberised cellulose | Typically in the range of | Typically in the range of |
| core | word pulp | 0.5-10 mm, e.g. 2-10 mm | 100-500 gsm, e.g. 250 gsm |
| | Super absorbent | | Typically in the range of |
| | polymer | | 0.5-3 g per sanitary pad, |
| | | | e.g. 1 g per sanitary pad |
| | Non-woven wet-laid | Typically in the range of | Typically in the range of |
| | cellulosic fibre | 0.15-1.15 mm, e.g. 0.6 mm | 5-80 gsm, e.g. 60 gsm |
| Sub layer | PVA | Typically in the range of | Typically in the range of |
| | | 0.5-60 μm, e.g. 0.8-12 μm | 1-80 gsm, e.g. 1-15 gsm |
| | | film | |
| Bottom | PVA | Typically in the range of | Typically in the range of |
| Sheet | | 5-60 μm, e.g. 30 μm film | 5-80 gsm, e.g. 40 gsm |
| | Non-woven wet-laid | Typically in the range of | Typically in the range of |
| | cellulosic fibre | 0.15-1.15 mm, e.g. 0.6 mm | 5-80 gsm, e.g. 60 gsm |

In a variant the first carrier layer 126 of the absorbent core 120 is omitted. Optionally a second layer of fiberised cellulose word pulp is included in its place. If so, the bonding sublayer 135 performs in the same way, to bond the PVA layer 144 of the bottom sheet 140 to the absorbent core 120, e.g. to a layer of fiberised cellulose word pulp.

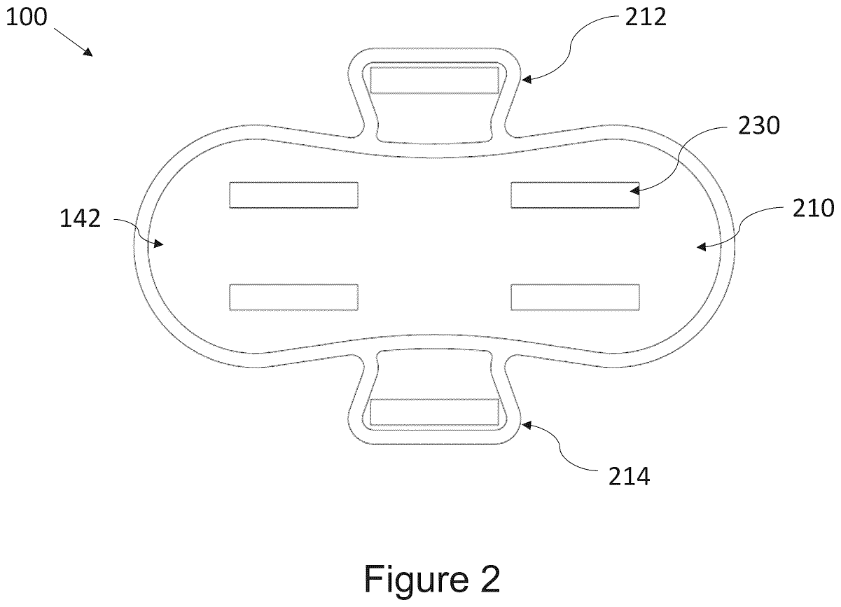

FIG. 2 shows a top down view of the sanitary pad 100. The geometric shape of the sanitary pad in this example is an oval main body 210 with opposing tabs 212, 214 extending to either side of the main body 210. The main body 210 may be oval, rectangular or waisted. The opposing tabs 212,214 are optional but can help in use to secure the sanitary pad to a surface with their positioning providing stability. In the illustrated example each tab is substantially trapezoidal, with rounded corners, but a variety of tab geometries can be used. The opposing tabs are removed by a distance C from the centre of the oval area. Alternatively the opposing tabs 212,214 are arranged centrally to the pad for a rotationally symmetrical shape. The tabs have a smaller inner length W than outer length X, reducing the length of the crease that occurs when the tabs are folded under the main body 210 of the pad. This crease is often associated with unwanted fluid leakage; reducing the length of the crease acts to reduce the possibility of unwanted leakage.

The tabs 212,214 of the sanitary pad 100 are formed from the top layer 110 and bottom sheet 140 extending to either side of the main body 210. The absorbent core 120 and sublayer 130 do not have tabs and are substantially oval in shape. The tabs 212,214 may alternatively be formed from the top layer 110, the bottom layer 140 or the bottom layer 140 and top layer 110 extending to either side of the main body 210. The sanitary pad 100 can also be provided without tabs with each layer being substantially oval in shape.

The layers of the sanitary pad 100 are bonded together around the perimeter of the main body 210 and tabs 212,214. The sanitary pad 100 is bonded together in this manner to secure the layers of the sanitary pad 100 together, to prevent moisture or fluid captured in the sanitary pad 100 from leaking out. In the case where only one layer forms the tabs then the bond can go around the oval main body 210, whereas if both layers form the tabs then the bond can go around the perimeter of the tabs.

The layers of the sanitary pad 100 are bonded together using a bonding technique for example using ultrasonic bonding which joins materials by way of frictional heat generated from high frequency (ultrasonic) mechanical motion. Under application of pressure and the frictional heat the PVA in the joint area can melt to create a bond between layers. A similar bonding can also be applied to sanitary pad 1100. However, the use of the hydrolysed PVA solution to bind the bottom sheet 140, the absorbent core 120 and the top sheet 110 together can provide a sufficiently strong bond between the absorbent core 120 and the bottom sheet 140 that means further bonding treatment is not necessary.

In use, the sanitary pad 100 absorbs menstrual fluid in the same way as a conventional sanitary pad. After use the sanitary pad 100 can be disposed of for example in a toilet. On contact with water in the toilet the soluble components of the sanitary pad dissolve and with the hydraulic action the insoluble components disperse. In particular soluble binders and PVA dissolve and allow the other components of the layers (such as cellulosic fibres) to disperse. It is noteworthy that the bottom layer 140 shows higher stability to blood than to water such as used in flushing. It is thought that this is due to the biological component of blood (platelets, red and white blood cells) and the volume of the solvent. The sanitary pad 100 is dispersible enough that normal flushing action of a toilet disperses and dissolves it sufficiently for it to pass through the sewage system. Cellulosic and PVA and CMC components of the sanitary pad are biodegradable and can be processed by conventional sewage treatment.

FIG. 2 shows the sanitary pad of FIG. 1 with adhesive attached to the exposed section of the bottom sheet 140. The adhesive pattern comprises several strips of adhesive 230 applied to the main body 210 of the bottom sheet 140. FIG. 2 also shows adhesive strips 230 applied to the tab sections 212,214 of the bottom layer 140 for securing the tab sections 212,214 to an undergarment.

The number of strips of adhesive 230 and the dimensions of the strips provided on the bottom sheet 140 are selected so that adhesion of the sanitary pad 100 to the undergarment prevents movement of the sanitary pad 100 during use, as well as avoiding areas where there is a high risk of moisture migration through the sanitary pad during use which may otherwise negatively impact the performance of the adhesive strips.

FIG. 2 shows four adhesive strips 230 secured to the main body 210 of the bottom sheet 140 and an adhesive strip 230 is provided on each of the tabs 212,214. Each adhesive strip 230 has a length of 50 mm and a width of 15 mm. The grams per square metre (gsm) of the adhesive strips is around 8-10 gsm dry weight. Other dimensions and arrangements of adhesive strips are possible and may be dependent on the size and shape of the sanitary pad. The adhesive used for the adhesive strips 230 is a water based pressure sensitive adhesive such as ST3007 from Scitech. Other water-based, water-soluble or water dispersible adhesives may be used as the adhesive.

Examples of properties of a water based pressure sensitive adhesive that is suitable for the adhesive strips are:

| | | Typical | Measurement |
|---|---|---|---|
| Characteristic | Units | Value | method |
| Viscosity | cP | Up to 10,000 | Brookfield RV at |
| | | or less, e.g. 250 | 20 C., spindle 2, |
| | | (+2000/−150) | speed 50 rpm |
| Solid content | % by weight | Up to approx. 50 | |
| | | or less, e.g. 46 | |
| pH | | 7-9 | |
| Loop tack | N/25 mm | 14 | at 18 gsm dry on |
| | | | glass |
| 24 h peel adhesion | N/25 mm | 10.7 | at 18 gsm dry on |
| | | | glass |
| Static shear | h | 23 | at 18 gsm dry on |
| | | | stainless steel |

As mentioned above sanitary pads are provided with a release liner. The release liner also acts as a wrapping which protects the sanitary pad 100 from damage and prevents dirt and moisture from contacting the sanitary pad 100 whilst the sanitary pad is not in use. A non-woven fabric with a film layer of PVA (applied e.g. by extrusion, thermal lamination or other means) is siliconized to form a suitable release liner. As the non-woven is porous, the PVA layer can provide an impermeable layer for the silicone to coat for advantageous performance. The non-woven cellulosic fabric has similar properties to those non-woven fabrics used in the first and second carrier layers. The non-woven fabric of the release liner could for example be a wet-laid cellulosic non-woven fabric such as those available under the tradename Hydraspun™ from Suominen, having a density of 60 gsm and a thickness of 0.6 mm. A layer of PVA, e.g. around 10-80 μm thick, may be applied to the non-woven fabric to form the release liner. A layer of silicone, e.g. around 1-4 gsm, may be applied to the layer of PVA to form the release liner.

An alternative water-dispersible release liner may be provided such as a paper-based liner laminated with PVA or a PVA liner may be used to protect the adhesive. A suitable water-dispersible release liner may be a hydrophobic coated PVA film and/or an embossed PVA film.

An example of the properties of a 80 μm thick paper-based liner (here a paper that includes a heat seal coating as is available under the trade name SmartSolve 3150A) that is suitable as a water-dispersible release liner are (at 23° C. and 50% relative humidity):

| Characteristic | Typical Values | | Measurement protocol |
|---|---|---|---|
| Elongation at Break | 5.5% | | ASTM D882 |
| Tensile Strength | 1.2 | kg | ASTM D882 |
| Weight | 50 | g/m² | |
| Density | 0.69 | g/m³ | |
| Dissolution time at 23° C. | 6 to 8 | s | IS 16154: 2014 |

An example of the properties of a 65 μm thick paper-based liner (similar to the above but without a heat seal coating) that is suitable as a water-dispersible release liner are (at 23° C. and 50% relative humidity).

| Characteristic | Typical Values | | Measurement protocol |
|---|---|---|---|
| Elongation at Break | 2.7% | | ASTM D882 |
| Tensile Strength | 0.9 | kg | ASTM D882 |
| Weight | 30 | g/m² | |
| Density | 0.46 | g/m³ | |
| Dissolution time at 23° C. | 6 to 8 | s | IS 16154: 2014 |

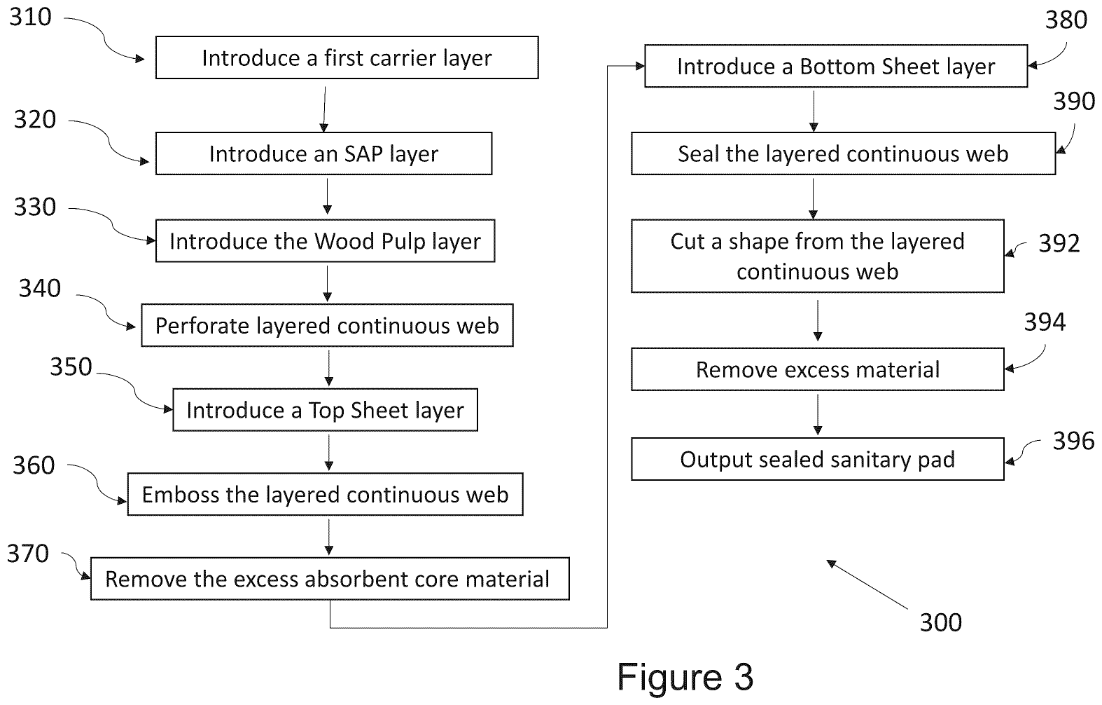

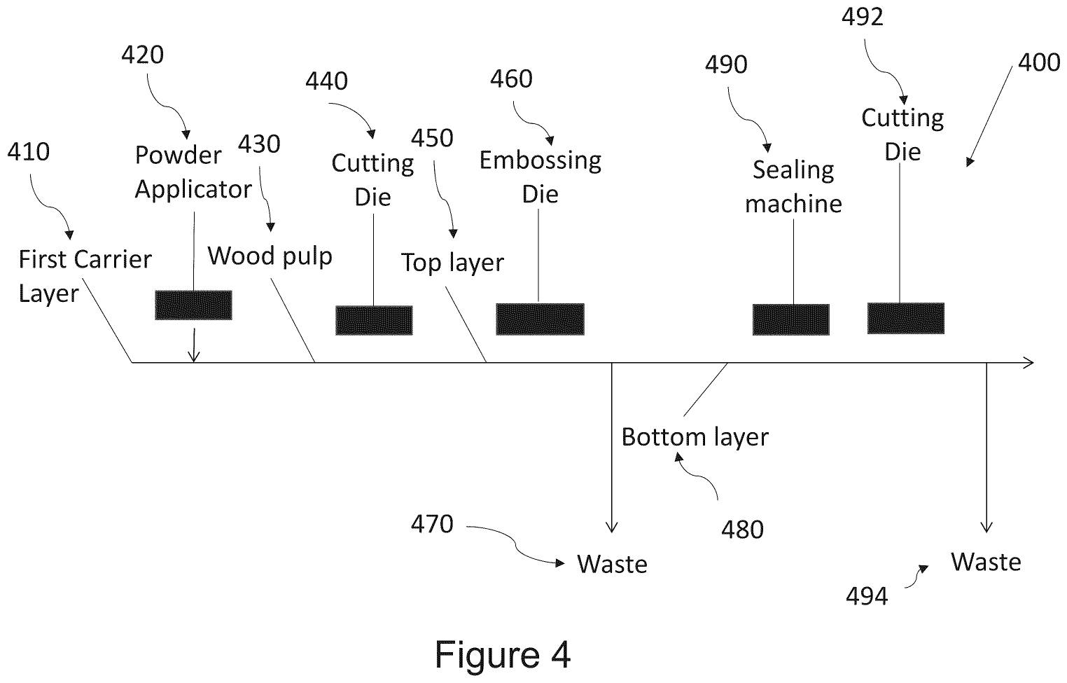

FIG. 3 shows a flow diagram 300 of a part of a process (through to outputting a sealed sanitary pad, prior to application of adhesive or release liner) for manufacturing water dispersible sanitary pads such as the sanitary pad that is shown in FIG. 1 and described above. FIG. 4 shows a schematic of part of an example assembly line for assembling sanitary pad 100.

It is common and desirable in the manufacture of textiles, and sanitary pads, to utilise continuous high speed manufacturing processes capable of forming a layered product from several different continuous webs of material. Rotary presses advantageously provide a high continuous throughput method of processing of continuous webs or sheets of materials. Such processing can permit web processing speeds of 75 m/min and 300 pads manufactured per minute, and the manufacturing processes described below are preferably adapted for such high continuous throughput methods.

Rotary presses commonly comprise two opposing rollers through which a continuous web or webs of material are passed. Rotary presses are commonly used to press and bond multiple layers of material together. For example, a continuous web of material may be pressed together with another continuous of material to produce a single continuous web. The single continuous web can then be processed further.

A difficulty often encountered in the high speed production of sanitary pads is slippage between the different layers of material during manufacture, in particular where rotary tooling is used. This results in the misalignment of layers during the manufacturing process and subsequent production of defective products. Conventionally this problem is addressed through the use of construction adhesives between layers. Construction adhesives adhere the layers of material together preventing the relative movement of the layers during processing. Conventionally construction adhesives are hot melt adhesives with a working temperature of 100° C. or higher such as synthetic rubber adhesives for example those available from Henkel under the tradename Technomelt. However, such construction adhesives are unsuitable or undesirable for flushable water-dispersible sanitary pads as they are not generally water soluble and therefore prevent the sanitary pad from dispersing in water.

FIG. 4 shows an assembly line 400 for a sanitary pad 100. A continuous web of carrier material 410 for example a non-woven cellulosic fibre is introduced to the assembly line 310. The continuous web of carrier material is then coated 320 with a layer of superabsorbent polymer for example a powdered cellulose gum. The powdered cellulose gum is applied onto the continuous web of carrier material in a homogenous layer to form a super absorbent polymer layer. The cellulose powder may be applied onto the carrier material; for example the carrier web may be passed through a continuous “curtain” powdered cellulose gum of a powder applicator 420. The powder applicator 420 comprises a mechanism that applies the cellulose powder onto the continuous web of carrier material, optionally from a number of different directions, to achieve a homogenous distribution of cellulose gum powder over the first carrier layer. The rate at which the web of carrier is passed through the continuous applicator may be varied to provide different thicknesses of superabsorbent polymer layer. In the illustrated example a powder spray applicator is used, but other methods of applying a homogeneously distributed layer of cellulosic gum powder on the first carrier may be used, such as a vacuum powder applicator.

A continuous web of fiberised wood pulp 430 is introduced 330 to the assembly and overlaid (married) the superabsorbent polymer coated continuous web of carrier material forming the basic structure of the absorbent core 120 (wood pulp layer 122, SAP layer 124, first carrier layer 126) described above.

The continuous layered web may at this point be overlaid on (married to) a continuous sub layer web 130. The sublayer 130 improves the performance of the sanitary pad 100 in the current example. The sublayer 130 is optional and may be omitted in other sanitary pads. The sub layer has a 20 μm PVA layer 132 and a second carrier layer 134 of non-woven wet laid cellulosic fibre of the type described above. The PVA layer may be extruded or otherwise applied (e.g. by thermal lamination or with a lamination adhesive) onto a continuous web of non-woven wet laid cellulosic fibre to achieve the continuous web of sublayer.

The layered continuous web is passed through a rotary press 440 with a cutting die.

The cutting die of the rotary tool 440 is provided in the shape of the main body 210 of the sanitary pad 100. The substrate moves through blades of the cutting die at a tangent to the rotary tooling and at a depth defined by the desired characteristics of the transformed substrate. In this manner the cutting die perforates the main body shape, in this example an oval shape, into the layered continuous web. The layered material is perforated 340 so that excess material from around the perforated shape may be more easily removed later in the manufacturing process, whilst ensuring sufficient integrity of the web to allow further processing without intra-layer slippage.

The cutting die thus pre-cuts absorbent cores out of a continuous web (in roll form or web form) of material (wood pulp and SAP and carrier). The perforation cut severs the shape around most of the shape perimeter, but not completely, so as to keep the continuous web sufficiently intact for further processing. This means that the cut core can be transported to the embossing stage without needing a complex indexing and leading-edge identification setup. The perforation cut can thus enable processability in the following stages of the process.

The degree of perforation may be varied to enable easy removal of the excess material or improve the structural integrity of the layer continuous web. An oval shape having a perimeter of which 95% is perforated has been found to provide sufficient structural integrity whilst allowing removal of excess material from around the perforated shape: the core shape stays attached to the web of material following the cut, but also can be removed with ease. Other levels of perforation are also possible for example 90%-99%. The level of perforation can be adapted depending on the tensile strength of absorbent materials, cutting process, the processing speed, the cutting blade details, and other processing factors. In an example a connecting portion (where the continuous web is not severed but remains intact) is arranged every 0.5-2 cm along the shape. In an example around 40-50, e.g. 48 connecting portions are arranged in an even distribution along the shape. In an example the connecting portions are evenly distributed around the perimeter of the oval shape, but in alternatives the connecting portions may be more densely positioned in certain areas. For example the connecting portions may be positioned to increase the web integrity in a particular direction, in particular in the direction of material pull. In another variant the perforations sever the carrier of the continuous web, but do not sever, or only partially sever, the wood pulp (or other fibre) layer of the continuous web. The wood pulp layer may have low structural integrity and may not require the same degree of perforation as the carrier layer in order to remove excess material around the perforated shape. The shape and dimensions of the connecting portions may also be varied to increase the integrity of the web in particular directions.

Alternatively cutting equipment may be used to perforate the layered web for example a continuously operating flat bed die cutting machine with a rule knife cutting tool may be used. As the continuous web is passed through the rollers or under the roller it is perforated a number of times at different positions, corresponding to the positions of the shapes perforated into the absorbent core and sublayer, along the length of the continuous web with the desired pattern and/or shape.

After the layered continuous web has been perforated a continuous web of top layer 450 material, in this example a non-woven cellulosic wet-laid spunlace material, is overlaid on (married to) the layered continuous web to form a layered continuous web 350 comprising a top sheet 110, wood pulp layer 122, SAP layer 124 and first carrier layer 126 and optionally the sublayer 130.

Following the marriage of the top layer 110, the layered continuous web is then passed through a second rotary press 460 having an embossing die. The layered continuous web is embossed with a pattern and/or shape 360. The embossment of the layered continuous webs secures the layers together and provides a fluid distribution pattern in the layered continuous web. Securing the layered continuous webs together prevents the different layers from displacing during the manufacturing process without the need for construction adhesive. The embossment can hold together the top layer and the absorbent cores against the force of removing the waste material outside the perforated shape. The pattern embossed in the top layer 110 forms channels to distribute fluid over the surface of the sanitary pad 100, enhancing absorption efficiency.

The embossing die 460 of the current example comprises a shape and a fluid distribution pattern, however an embossing die having another shape or a fluid distribution pattern may be used. The embossing die 460 may include male and female embossing counterparts, or a male roller with a flat anvil counterpart.

In the current example, an oval shape having a fluid distribution pattern is embossed into layered continuous web, the layered continuous web is indexed or otherwise aligned in the rotary embossing press 460 such that the oval shape is embossed into the section of the layered continuous web comprising the perforated shape. The embossed shape may alternatively comprise an oval shape with tabs extending from the central region to produce an embossed shape in the layered continuous web that corresponds to the shape of the sanitary pad described above.

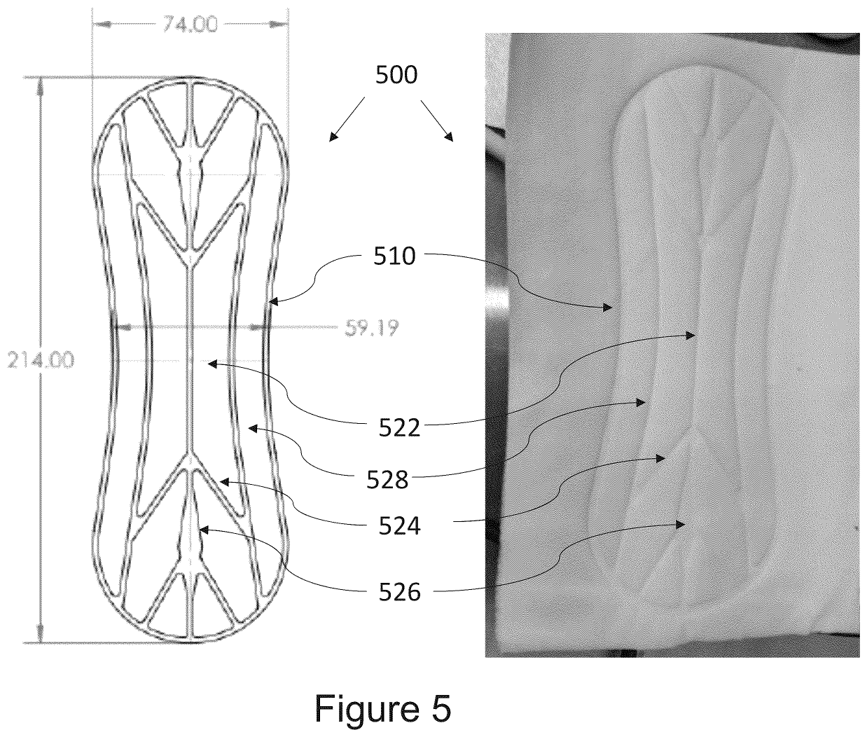

FIG. 5 shows a top view of a fluid distribution pattern 510 embossed into a layered continuous web. In the illustrated example the pattern of embossments is formed of a series of parallel longitudinal channels 520 extending along the length of the oval shape 510. A central longitudinal channel 522 has along its length a number of pairs of diagonal lines 524 extending symmetrically from the central channel 522 to connect the central channel 522 to adjacent longitudinal lines 528 or the perimeter of the shape 510. The width of the central line channel is varied at positions along the length, in the example of FIG. 5 the channel increases in width toward the ends of the device to form two wide sections 526. In each case the central channel 522 then narrows before reaching the perimeter 510 of the sanitary pad 100. A variety of alternative patterns may be suitable; for example, the pattern may comprise fractal patterns, geometric patterns of repeating rhomboidal shapes and/or longitudinal and transverse lines across the sanitary pad.

The embossments may extend through the top layer 110 and partially into the absorbent layer 120. A bond (such that the bond strength is sufficient for downstream processing) is formed between the top layer 110 and the absorbent layer 120 at the embossments. The embossments in the top layer 110 can help reduce skin contact of fluid, and so give a dryer feel in use. The embossments also help constrain movement of the underlying absorbent layer by forming pockets of absorbent material and so helping to avoid so-called core-slipping. The embossments also improve the transfer rate of fluid from the top layer 110 to the absorbent core 120. Embossments in the top layer 110 can provide better performance than perforations, as perforations can reduce structural integrity of the sanitary pad 100 unfavourably.

As the layered continuous web is passed through or under the roller 460 the embossing dies of the rotary press emboss the material with the desired shape and/or pattern 360. The embossments are aligned with the perforated shape made in the continuous web of absorbent core 120 and sublayer 130. As the continuous web is passed through the rollers or under the roller it is embossed a number of times at different positions, corresponding to the positions of the shapes perforated into the absorbent core and sublayer, along the length of the continuous web with the desired pattern and/or shape.

Following the embossing of the layered continuous web 360 excess absorbent core 120 and sublayer 130 material is removed from the layered continuous web. The excess absorbent core material and sub layer material 470 surrounding the perforated shape is removed 370 and once removed the layered continuous web has an embossed top sheet 110 and oval shaped absorbent core 120 and sub layer 130. The perforations of the absorbent core 120 and sublayer 130 provide weak points at which these layers preferentially tear, assisting the process of the removal of excess from material from the layered continuous web. The embossments of the layered continuous web secure the top sheet 110, absorbent core 120 and sublayer 130 together and help prevent relative movement of the top sheet 110, absorbent core 120 and sublayer 130 during this removal process. The combination of the perforating the absorbent core 120 and embossing the layered continuous web has a particular beneficial and synergistic effect on this removal process in that a lower force is required to remove the excess material because of the perforations and the embossments provides a sufficiently strong bond between the top layer and the absorbent core to prevent relative movement during this stage. Conventionally, construction adhesives are used to prevent relative movement of the layers of the sanitary pad but the current process that utilises perforation and/or embossing does not require construction adhesives.

The layered continuous web is then overlaid 380 on (married to) a continuous web of bottom sheet 480 of the type described above. A bottom sheet 140 as described above comprises a reinforced wet-laid cellulosic non-woven fabric with a 30-50 μm PVA film extruded onto its surface.

The layered continuous web is then passed through a continuous sealing machine 490. The sealing machine 490 seals the perimeter of a shape into the layered continuous web. The sealing shape comprises the oval shape 210 of the absorbent core 120 and optional sublayer 130 and in addition the sealing shape comprises the shape of the tabs 212,214 extending from the oval shape that is described in detail above. The sealing 390 of the perimeter of the oval shape seals together all the layers of the layered continuous web. The layered continuous web is aligned in the sealing machine such that the oval shape of the sealing machine aligns with the oval shape of the absorbent core 120 and optional sublayer 130. The sealing of the perimeter of the tabs 212, 214 shapes seals together the top sheet 110 and the bottom sheet 140 and forms the tabs of the sanitary pad 100.

The layers of the sanitary pad 100 are bonded together and sealed using a bonding technique for example using ultrasonic bonding which joins materials by way of frictional heat generated from high frequency (ultrasonic) mechanical motion. Under application of pressure and the frictional heat the PVA in the joint area can melt to create a bond between layers.

In a simple example ultrasonic bonding is implemented with a suitably shaped horn or a sonotrode that can be pressed against an anvil on which the unbonded layers to be bonded can be arranged to cut and weld the layers together. For example, for a rotationally symmetrical sanitary pad shape a horn or sonotrode is shaped so as to seal a half of a pad and then the substrate is rotated to seal the other half. As the surface area of the bond surface is large, for satisfactory bonding only half the pad is bonded per sonotrode and stroke. In another example a horn or sonotrode is provided that is shaped to provide a seal around the pad. Pressing the horn or sonotrode down onto the unbonded layers permits bonding of the layers to form a pad in a single work stroke, without repositioning the pad. In another example continuous sealing is provided by a suitably formed sonotrode drum that rotates against a suitable anvil. As the layered continuous web is fed into the sonotrode drum, the sonotrode drum rotates and bonds the pad.

Sealing 390 of the sanitary pad 100 prevents fluid from leaking out from the sanitary pad 100 and in particular the absorbent core layer 120 and secures the layers of the sanitary pad 100 together.

Following the sealing of the sanitary pad the layered continuous web is passed through a rotary press with a cutting die 492. Alternatively a continuously operating flat bed die cutting machine with a rule knife cutting tool may be used.

The cutting die 492 of the rotary press is provided in the shape of the desired shape of the sanitary pad 100, in this case the oval central section 210 with the tabs 212,214 extending from the centre, and is pressed into the layered continuous web to cut the shape into the layered continuous web 392. The layered continuous web is indexed or otherwise aligned in the rotary press such that the cutting die shape is cut into the section of the layered continuous web comprising the sealed shape such that a sealed sanitary pad is cut out of the continuous layered web.

The excess material 494 comprising mainly the material of the top 110 and bottom sheet 140 around the cut shape is removed 394 to leave 396 a sealed water-dispersible sanitary pad 100 having the structure and properties described above and not containing construction adhesives that are conventionally used in the manufacture of sanitary pads.

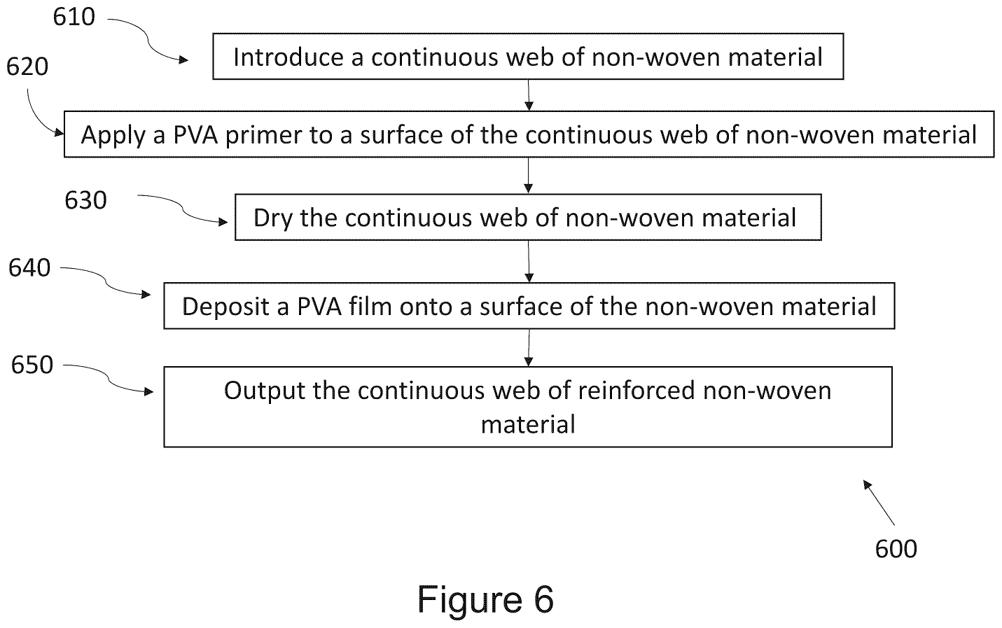

FIG. 6 shows a method 600 of manufacturing a reinforced wet-laid cellulosic non-woven fabric.

As described above in relation to sanitary pad 100 it is desirable to reinforce the wet-laid cellulosic non-woven fabric of the bottom layer of the sanitary pad. The reinforcement of the non-woven fabric of the bottom layer with an extruded PVA film advantageously improves the adherence of the adhesive to the non-woven fabric and also improves the structural integrity of the non-woven fabric. In particular reinforcement of the non-woven fabric improves the structural integrity of the sanitary pad 100 during the removal of sanitary pad from an undergarment and prevents damage to the bottom layer ensuring a clean removal of the sanitary pad and pressure sensitive adhesive from the undergarment.

To improve the performance of the bottom layer a continuous web of the non-woven material is introduced 610 to a manufacturing process and first a primer is applied, the primer being PVA (or alternatively CMC) dissolved in water 620. The primer is for application of an ultra-low gsm (e.g. in the range of 1-5 gsm or <1 gsm) quantity of water-soluble polymer to the non-woven fabric, so as to form a thin coat 142 of PVA (or CMC) on the non-woven fabric, and serves to promote adhesion of an adhesive to the bottom layer. In an example the primer is 2% by weight PVA in water; the primer is typically about 0.1-5% by weight polymer in water, though higher concentrations can serve too, e.g. up to 15%. Other solvents may be used. High hydrolysis PVA solutions as discussed above can be particularly effective for priming, as high hydrolysis PVA can be less sensitive to cold water and perform well as a binder. A thin film of primer is applied to form a coating 142 for example by passing the continuous web of material continuously through a spraying rig comprising a number of spraying nozzles that spray the primer onto the continuous web. The spraying nozzles may be arranged in the spraying rig at a number of different positions so that the non-woven fabric is sprayed with the primer from a number of different directions. Other methods may be used to apply the primer; for example gravure or slot die methods of application may be used. Following the priming of the non-woven material, the continuous web is then dried 630 to evaporate the water from the primer. For example the continuous web is passed through a drying tunnel that comprises a number of air blowers (preferably hot air blowers) that continuously blow hot air onto the primed non-woven material to dry the material by evaporating water from the non-woven material. Other conventional approaches for drying may be used, for example involving heaters or irradiation.

Following the drying of the non-woven material, PVA is extruded 640 onto the opposite surface of the non-woven material to form a PVA film, preferably a 30-50 μm film. The PVA film may be extruded on the surface of the non-woven material for example using a slot die. Other methods of depositing or forming the PVA film may be used. The manufacturing process 600 outputs the reinforced non-woven material 650.

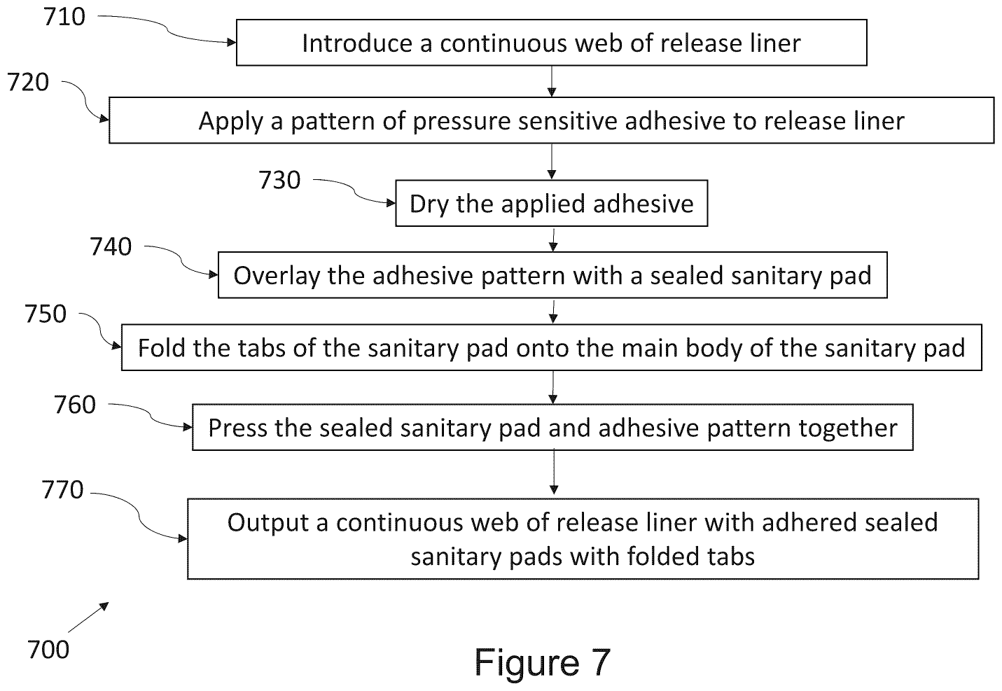

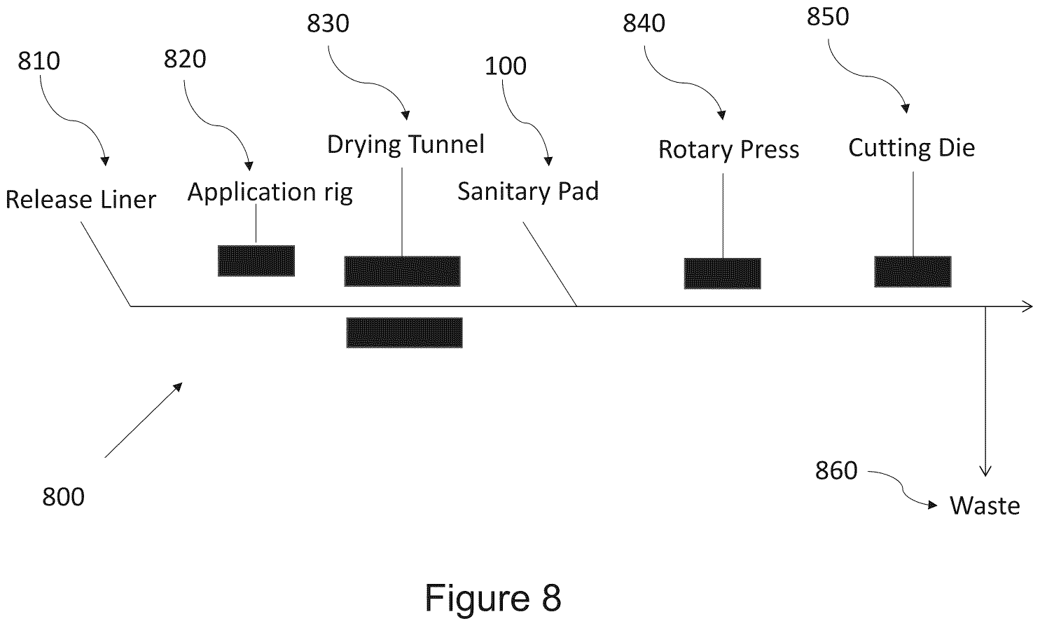

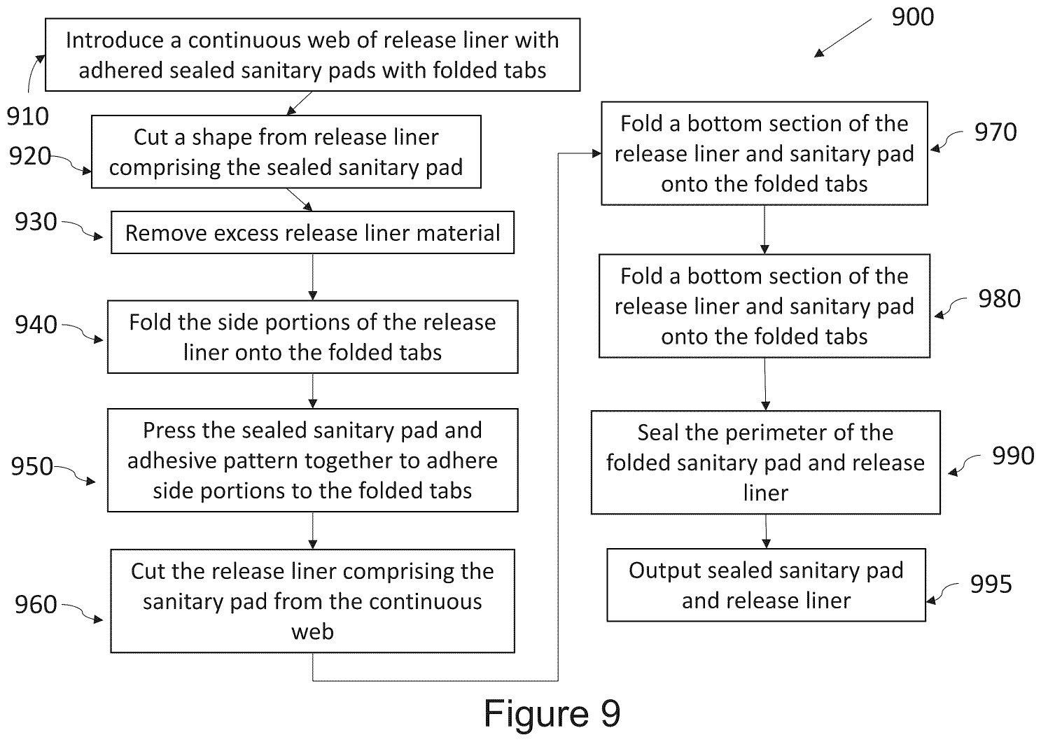

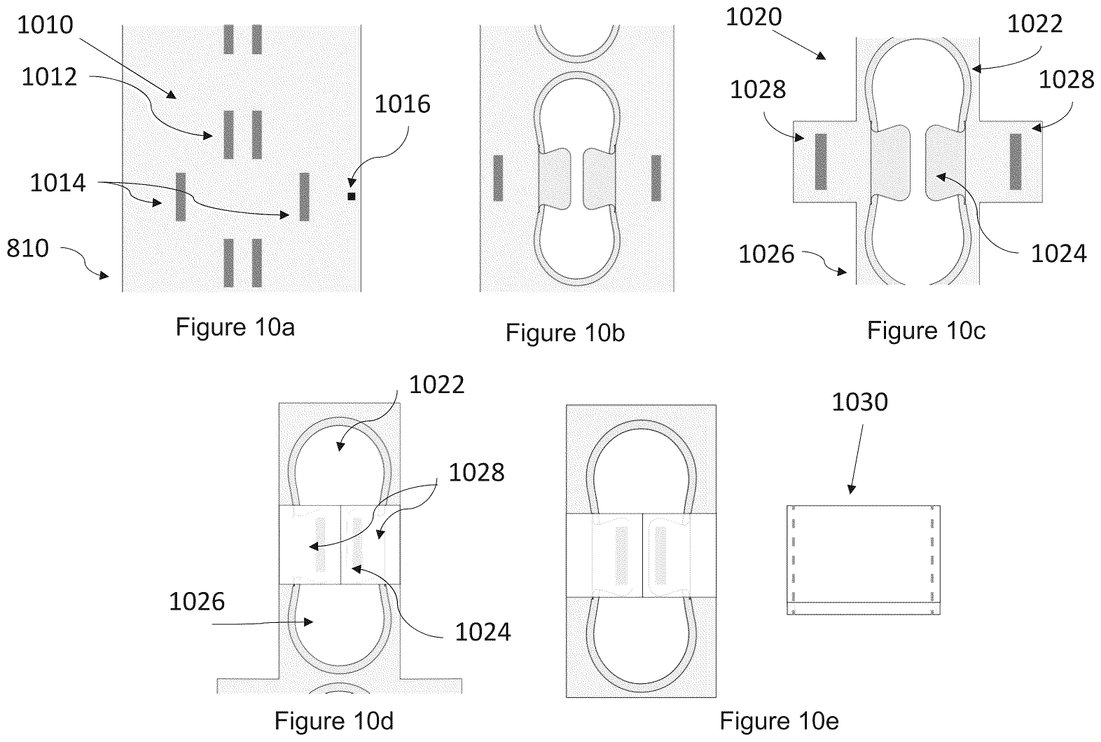

FIG. 7 shows a flow diagram of a method of manufacturing a release liner having an adhesive pattern for the sanitary pad 100 that is shown in FIGS. 1 and 2 and described above. FIG. 8 shows a schematic of part of an example assembly line 800 for assembling sanitary pad 100. FIG. 9 shows a flow diagram of a method 900 of assembling and packaging a sanitary pad 100. FIGS. 10a to 10e show a sanitary pad at various stages of the assembly illustrated in FIG. 9.

A continuous web of non-woven fabric with PVA film is siliconized to form a suitable release liner 810. The non-woven cellulosic fabric can have similar properties to the non-woven fabrics used in the first and second carrier layers as described above. The non-woven fabric of the release liner could for example be a wet-laid cellulosic non-woven fabric such as those available under the tradename Hydraspun™ Royal from Suominen, having a density of 60 gsm and a thickness of 0.6 mm. Other materials such as those described above may be used for the release liner and in the following process of applying adhesive material. For effective release performance coating with a silicone is preferred. Coating silicone directly onto the non-woven fabric can exhibit poor surface hold. To overcome this a PVA film can be applied, e.g. by lamination, to the non-woven fabric to create a sealed surface for the silicone. In an alternative a priming process, e.g. as described above, is used to apply a low quantity of water-soluble polymer (typically PVA or CMC) to the non-woven fabric, so that the polymer acts as a sizing agent for the silicone and seals the non-woven surface.

The continuous web of siliconized non-woven is passed through a spraying rig 820 and a pattern of adhesive is sprayed on a surface of the siliconized non-woven 720. Siliconizing the non-woven protects the non-woven from the pressure sensitive adhesive which may have adverse effects on its structural integrity. The adhesive is a biodegradable water based pressure sensitive adhesive for example the water based pressure sensitive adhesive available under the trade name ST3007 from Scitech. The adhesive can be applied using spray valves that atomise the water based pressure sensitive adhesive using high-pressure forced air through an atomising aperture. The pattern of adhesive applied to release liner comprises several strips arranged such that the positions of the strips correspond to areas of the sanitary pad 100. The adhesive strips are positioned such that a sanitary pad 100 may be overlaid onto the adhesive pattern and the adhesive strips adhered to the main body 210 (oval region) and tab sections 212,214 of the sanitary pad 100. FIG. 10a shows an example of such an adhesive pattern 1010 on the release liner 810 wherein the pattern comprises four adhesive strips 1012 forming a rectangular shape dimensioned such that when overlaid with a sanitary pad 100 the four strips of adhesive adhere to the main body 210 (oval section) of the sanitary pad 100. The adhesive pattern 1010 comprises two adhesive strips 1014 to either side of the rectangular shape 1012 and dimensioned such that when the adhesive pattern is overlaid with a sanitary pad the two strips 1014 of adhesive adhere to the tab 212,214 sections of the sanitary pad 100. Other adhesive patterns may be implemented.

As the continuous web of release liner 810 is passed through the spraying rig 820 adhesive patterns 1010 are applied, such that the release liner 810 comprises several adhesive patterns 1010 along its length. The adhesive patterns 1010 are separated by a gap to enable a sanitary pad 100 to be overlaid onto each of the adhesive patterns 1010 and also to allow a cutting die 850 to cut around each adhesive pattern 1010. Alternatively the adhesive may be applied to the release liner 810 using a different method for example by direct coating using an aperture open to the release liner giving a direct interface between the release liner and the pressure sensitive adhesive. A fluid restrictor may be used in combination to achieve a given thickness of the applied water based pressure sensitive adhesive on the release liner. Gravure techniques or slot die applications may be used to apply the adhesive.

Following the application 720 of the adhesive, the release liner 810 is passed through a drying tunnel 830 to dry the adhesive 730. The drying tunnel 830 comprises a number of drying elements for example heaters or air blowers, preferably hot air blowers that continuously blow hot air onto the release liner to dry the water based pressure sensitive adhesive. Preparing the adhesive in this manner improves the adhesion of the pressure sensitive material to a bottom layer of a sanitary pad 100. In particular, it prevents the water-based pressure sensitive material from adversely affecting the non-woven material used in the bottom layer 140 and/or the PVA film provided on a reinforced non-woven material bottom layer 140. However, the ability to adhere to the bottom layer 140 is balanced with having an overly strong adhesive that causes in use on removal from an undergarment for the sanitary pad to tear or undergarment garment to tear, another undesirable characteristic of the product. As described above, primer treatment of the exposed surface of the bottom layer and formation of a thin PVA film improved cohesion between the adhesive and the bottom layer 140. The drying rate and/or thickness of the water based adhesive can affect the strength of the adhesion between the bottom layer and the water based adhesive and can therefore also be used as parameter to prevent undesirable tearing of the sanitary pad or undergarment. The thickness of the adhesive can be controlled by the rate at which the spray nozzle deposits the pressure sensitive adhesive onto the release liner and/or the rate at which the release liner is passed through the spraying tunnel. The drying rate of the adhesive may be varied by changing the rate at which the release liner is passed through the drying tunnel and/or the temperature or strength of air blowers in the drying tunnel. It is observed that with the process of applying adhesive to the release liner, and then transferring adhesive from the release liner to the bottom layer of the sanitary pad a suitably adhesive sanitary pad can be manufactured with a similar peel strength as is conventionally provided in sanitary pads.

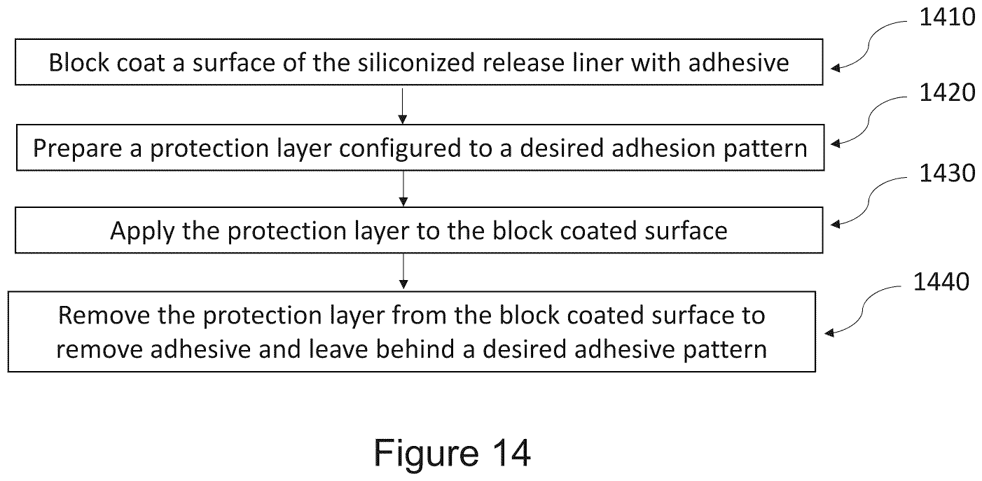

FIGS. 12, 13 and 14 illustrate an alternative method of forming an adhesive pattern 1010 onto the release liner 810. A surface of the release liner 810 is block coated (i.e. coated uniformly over the surface without patterning) with an adhesive 1230. The adhesive is a biodegradable water based pressure sensitive adhesive for example as described above. Water based pressure sensitive adhesives can be particularly challenging to pattern, as they may have high sensitivity to shear and may not be suitable for patterning techniques such as gravure coating. In particular the use of block coating can enable use of an adhesive that may not be suitable for processing methods involving shear, as patterning approaches such as spray application or gravure application. The block coating of adhesive is applied at around 8-10 gsm dry weight in an example.

A protective sheet 1200 is provided. An example of a protective sheet is shown in FIGS. 12 and 13. The protective sheet 1200 includes portions 1220 that can adhere to the block coated adhesive, and other portions 1210 that do not adhere well to the block coated adhesive, patterned according to the desired adhesive pattern. In the example shown in FIG. 12 the protective sheet 1200 is patterned to form a 3×2 array of rectangular adhesive strips, with the portions 1210 that do not adhere well corresponding to where the adhesive is desired. The protective sheet 1200 is a sacrificial sheet that does not form part of final product but assists in the patterning of the block coating of the adhesive.

In an example, the protective sheet 1200 includes non-siliconized areas 1220 and siliconized areas 1210. The siliconization may be applied to the film using a suitable technique for example, spraying, slot die, gravure etc. In another example the protective sheet 1200 is formed with help of a mask; the mask is placed over a uniformly siliconized sheet and then corona treatment is applied to de-siliconize exposed areas of the sheet. In another example the protective sheet 1200 is formed by cutting and removing portions of a non-siliconized sheet; optionally a siliconized sheet may be arranged behind the thus formed apertures in the non-siliconized sheet.

As the protective sheet 1200 is only used in manufacturing but not included in the final product it can be selected from a wide range of materials and need not be limited to flushable materials.

The protective sheet 1200 is applied 1430 to the block coating of adhesive 1230 on the surface of the release liner 810, for example, by overlaying the protective sheet 1200 onto the adhesive 1230. The non-siliconized parts 1220 of the protective film 1200 can bond with the adhesive 1230, whereas the siliconized parts 1210 of the protective film 1200 do not bond well with the adhesive 1230. When the protective sheet 1200 is removed those parts of the block adhesive that are in contact with the siliconized parts 1210 are pulled away from the release liner 810, and those parts of the block adhesive that are in contact with the non-siliconized parts 1220 are not pulled away and instead remain on the release liner 810. of the block adhesive, and the block adhesive remains in the areas in contact with the siliconized parts 1210 of the protective sheet 1200. The protective sheet 1200 with the superfluous adhesive can be removed and disposed of.

In an alternative method of forming an adhesive pattern on the release liner, the release liner is patterned to comprise adherent portions and non-adherent portions (with the release liner otherwise as described above). An adhesive is block coated (i.e. coated uniformly over the surface without patterning, e.g. as described above) onto the thus prepared release liner. The adherent portions of the release liner can adhere to the block coated adhesive, and the non-adherent portions of the release liner do not adhere well to the block coated adhesive. The adhesive is a biodegradable water based pressure sensitive adhesive for example as described above.

In an example, the release liner is patterned to include non-siliconized areas and siliconized areas. The siliconization may be applied to the film using a suitable technique for example, spraying, slot die, gravure etc. In another example the release liner is formed with help of a mask; the mask is placed over a uniformly siliconized sheet and then corona treatment is applied to de-siliconize exposed areas of the sheet.

When, in use, the release liner is removed those parts of the block adhesive that are in contact with the siliconized parts are pulled away from the release liner, and those parts of the block adhesive that are in contact with the non-siliconized parts are not pulled away and instead remain on the release liner. The release liner with the superfluous adhesive can be removed and disposed of.

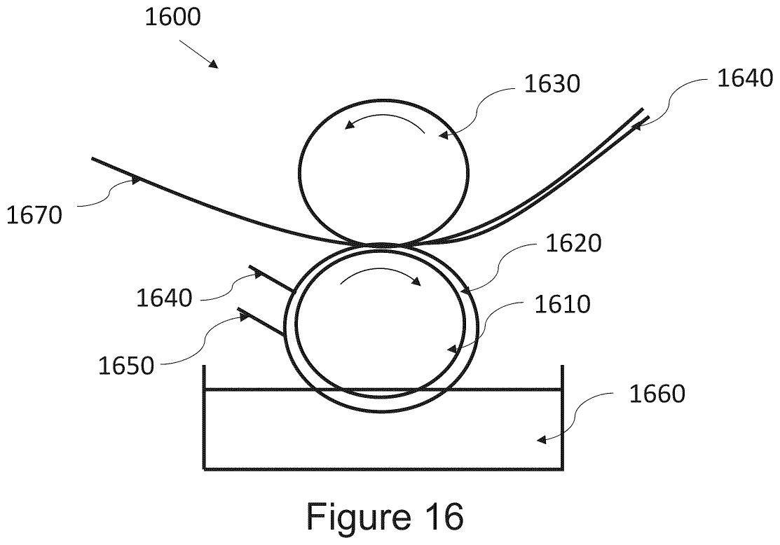

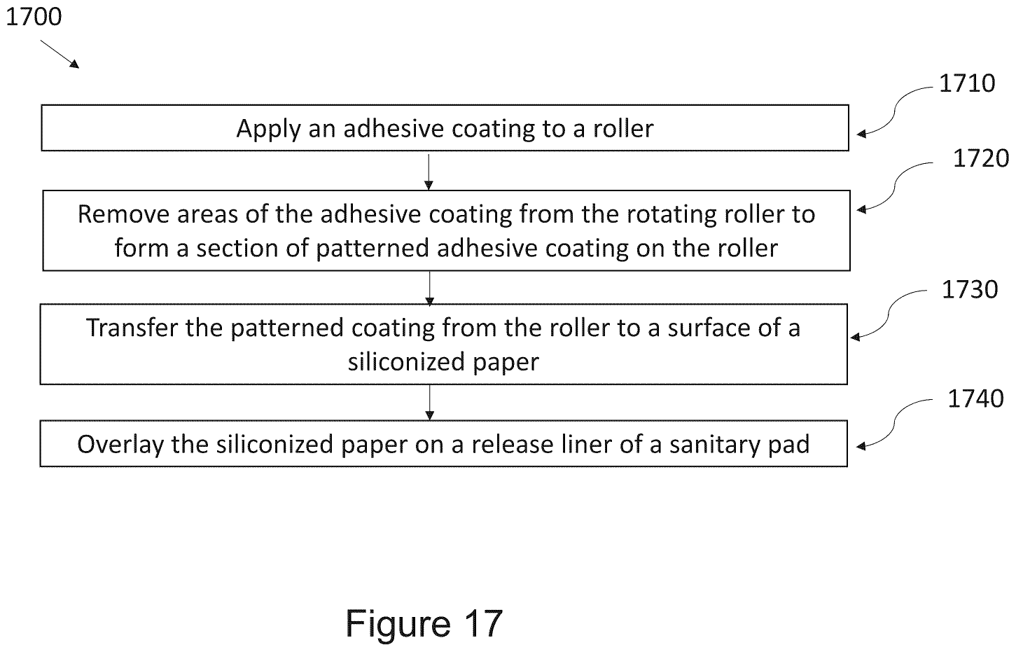

FIG. 17 describes another method 1700 for applying an adhesive pattern to a release liner in particular a release liner comprising a non-woven material such as release liner 810 described above. An example apparatus 1600 for carrying out the method 1700 is shown in FIG. 16.

In more detail, the method 1700 for applying an adhesive pattern to a release liner uses a roller 1610 coated 1710 with a layer of adhesive 1620. The adhesive may be one of the water based pressure sensitive adhesives described in detail above. A section of the coating of adhesive on the roller 1610 is patterned 1720 using one or more patterning tools 1640. The one or more patterning tools 1640 contact or are in close proximity to the surface of the roller 1610 and are arranged such that when the roller 1610 is rotated the patterning tools 1640 remove sections of the adhesive coating 1620 on the roller 1630 so as to form a patterned coating on at least a section of the roller 1610. The adhesive coating 1620 may not need be completely removed from the roller 1610 to form a patterned adhesive, it may also be sufficient to thin the adhesive coating in the desired areas to form a patterned adhesive section.

The one or more patterning tools 1640 may for example be a doctor blade or another suitable tool and are not limited to having a particular blade or tool profile. Doctor blades having a rectangular tooth profile or other gaps in a suitable arrangement can for example pattern the adhesive into strips (running in machine direction) of a suitable width and spacing. The patterning tools 1640 may also be automated to move in relative to the surface of roller 1610 thereby allowing different patterns to be formed in the adhesive coating 1620. Other non-patterning tools 1650 may also be employed to regulate the thickness of the adhesive coating on the roller 1610.

The patterned coating is transferred 1730 at least in part from the roller 1610 to a siliconized paper 1670 for example a glassine or clay paper. In the example shown in FIG. 16 the patterned coating is transferred from the roller 1610 to the siliconized paper 1670 via a reverse rolling process. In more detail, a feed roller 1630, is rotated in an opposite direction to the coated roller 1620. The feed roller 1630 feeds the siliconized paper 1670 past the patterned coating section created by the patterning tools 1640 on the surface of the rotating roller 1610. As the siliconized paper 1670 moves past the patterned adhesive coating, the patterned adhesive coating is at least in part transferred by contact from the roller 1610 to the siliconized paper 1670. The patterned adhesive is transferred from the roller 1610 to the siliconized paper 1670 in the areas where the adhesive coating has not been removed or thinned by the patterning tools 1640. The coating on the roller 1710 is preferably continuously replenished with adhesive, for example by rotating a part of the roller 1610 through an adhesive reservoir 1660. The transfer of adhesive can be used in a continuous manufacturing process such as that shown in FIG. 16, in which an adhesive pattern is continuously transferred to a continuous sheet of siliconized paper via a reverse rolling process.

Instead of or in combination with the doctor blade coating described above reverse roll coating can be used to transfer of the patterned adhesive to a surface of the siliconized paper 1670. In brief, in reverse roll coating a metering roll and an applicator roll with a gap between them counter-rotate (i.e. they rotate such that at the gap between them the surfaces move in opposite directions). The surface of the applicator roll is loaded with an excess of coating prior to the gap and as the rolls counter-rotate the applicator roll emerges from the gap with a layer of adhesive. The applicator roll can then transfer this layer of adhesive to the siliconized paper 1670. By providing a metering roll with a profile with a number of gaps the adhesive can be patterned into strips (running in machine direction) of a suitable width and spacing.

Following the transfer of the patterned adhesive to a surface of the siliconized paper 1670, the patterned adhesive on the surface of the siliconized paper 1670 may then be dried, for example using a drying tunnel or other drying methods described above. Then the patterned adhesive on the surface of the siliconized paper 1670 is overlaid onto a release liner, for example the release liner 810 described above, of a sanitary pad such that the patterned adhesive surface of the siliconized paper 1670 is in contact with the surface of the release liner—in the case of release liner 810 the siliconized paper is overlaid such that the patterned adhesive surface is in contact with a surface of the siliconized non-woven fabric of the release liner.

The overlaying of the siliconized paper 1670 and the release liner 810 can be performed in a continuous manner in which a continuous sheet of siliconized paper 1670 having a patterned adhesive on a surface is overlaid onto a continuous web of release liner material. For instance, in a lamination process (e.g. with an additional roll brought in on rewind) the adhesive transfers to the release liner. A variety of transfer coating processes can be used, for example.

The siliconized paper is then removed to leave behind a patterned adhesive on the surface of the release liner. The patterned release liner can be combined with a sanitary pad such as sanitary pad 100 or 1100 to form a sanitary pad product. Details of how the release liner and a sanitary pad might be combined are described in detail below.

The siliconized transfer paper 1670 need not be flushable, as appropriate disposal otherwise can be ensured. This can be beneficial as the release liner can be sensitive to moisture, as it is designed to be flushable. The described method 1700 can enable avoidance of application of a wet water-based adhesive directly to the release liner and avoidance of subjecting the release liner to drying, both of which can lead to damage and irregularities in the release liner.

The methods of applying a patterned adhesive can be used in combination with the other methods described herein. In particular they can form part of continuous production process for a sanitary pad product such as sanitary pad 1000 or 100. For example, these methods could be used in step 720 of method 700 wherein a pattern of pressure sensitive adhesive is applied to a release liner.

For overlaying a sanitary pad 100 onto the adhesive pattern 1010 a high speed vision system or sensor may detect the adhesive for alignment of the layers. The release liner 810 may be indexed with a readable index marker 1016. The index marker or markers indicate the positioning of the adhesive pattern 1010 or patterns so that a sanitary pad 100 may be overlaid on to the adhesive pattern. Alternatively once initial alignment is achieved the rotary elements may synchronize using highly accurate servo motors so as to maintain alignment.

A continuous web of release liner 810 comprising adhesive patterns 1010 and associated index markers 1016 such as the release liner described above is introduced into an assembly line.

As the release liner 810 is passed along the assembly line the indices 1016 of the release liner are read to indicate the positioning of an adhesive pattern 1010. A sanitary pad such as the sanitary pad 100 produced by the manufacturing process described above is deposited onto the adhesive pattern 740. The sanitary pad 100 is deposited onto the release liner and the tab sections 212,214 of the sanitary folded 750 over onto the main body 210 (oval portion) of the sanitary pad 100. The tabs may be folded over using a folding machine prior to the introduction to the assembly line or may be folded over after the introduction of the sanitary pad 100 onto the release liner 810. The bottom layer 140 of the sanitary pad 100 is positioned directly on top of the adhesive pattern 1010 and the four adhesive strips 1012 that form the rectangular shape of the adhesive pattern are overlaid by the oval section 210 of the sanitary pad 100. The two adhesive strips 1014 to either side of the rectangular shape are not at this stage overlaid with the sanitary pad 100 as the tabs 212,214 are folded onto the main body.

The deposited folded sanitary pad 100 and continuous web of release liner 810 is then passed through a rotary press 840. The rotary press 840 presses the deposited folded sanitary pad 100 and release liner together 760. The pressure from the rotary press causes the adhesive strips 1010 overlaid by the main body 210 (oval section) of the sanitary pad 100 to adhere to the bottom layer 140 of the sanitary pad 100.

Following the rotary press 840 the release liner 810 and folded sanitary pad 100 are passed through a rotary press with a cutting die 850. The cutting die 850 cuts out portions 920 of release liner 810 comprising the sanitary pad 100 and the adhesive pattern 1010 from the continuous web of release liner 810. In this example, a cross shape 1020 shown in for example in FIG. 10c is cut into the release liner. The cross shape 1020 comprises a top section 1022, central section 1024, bottom section 1026 and two side (wing) sections 1028. The top section 1022 and bottom sections 1026 remain connected to the continuous web of release liner 810; for example the top section 1022 connects to another bottom section of another cross shape further along in the assembly line and the bottom section connects to the release liner further behind on the assembly line that may not have been cut by cutting die 850. The cross shape 1020 is larger in area than the folded sanitary pad 100 and the adhesive pattern 1010 such that when the cross shape 1020 is cut from the continuous web of release liner 810 it comprises the adhesive pattern 1010 and folded sanitary pad 100 and also includes a perimeter of release liner material 810 extending beyond the sanitary pad 100 and the adhesive pattern 1010. The perimeter of release liner material is utilised later in the process allowing a portion of the release liner that may be sealed to easily seal the sanitary pad and release liner.

Alternatively cutting equipment may be used to cut the release liner; for example a continuously operating flat bed die cutting machine with a rule knife cutting tool may be used.

The excess release liner is then removed 930 from the assembly line so that a cross shaped release liner 1020 comprising the adhesive pattern 1010 and the sanitary pad 100 remains.

The two adhesive strips 1014 and the associated side sections 1028 of release liner 810 are then folded 940 over to adhere to the bottom layer 140 of the folded tabs 212,214 of sanitary pad 100. The folded sanitary pad 100 and folded release liner 810 may then be passed through 950 a rotary press 850 to adhere the adhesive strips 1014 of the side sections 1028 of the release liner 810 to the folded tab sections 212,214 of the sanitary pad 100 and improve the adhesion of the adhesive strips 1014 to the main body of the sanitary pad 100.

The sanitary pad 100 and attached release liner 810 are then cut 960 from the continuous web of release liner 820 by cutting die 850 and the cross shaped release liner 1020 and associated sanitary pad 100 folded and sealed to produce a final product. In particular the sanitary pad 100 and release liner 810 are tri-folded as shown in FIG. 10e. The bottom section 1026 of the cross shape 1020 is folded 970 into the centre 1024 of the sanitary pad 100 to cover the folded tab sections 212,214. The top section 1022 of cross shape 1020 is then folded 980 into the centre 1020 of sanitary pad 100 to cover the folded bottom section 1026. The release liner 810 is then sealed 990 using a heat seal or ultrasonic seal for example along the perimeter of the release liner 810 that extends beyond the folded sanitary pad 100. A sealed and folded sanitary pad and release liner 1030 is output 995 from the assembly line. This method of folding the sanitary pad and attached release liner has been found to be particular effective at preventing foreign particulates from reaching the top sheet of the sanitary pad. This is particularly important as the sanitary pad is often stored and transported before use for example in a handbag.

In a variant of the sanitary pad described above, an acquisition distribution layer (ADL) is included. It is known to include ADLs between the sandwiched between the top sheet and the absorbent core to aid with fluid distribution in the pad and to prevent fluid accumulation at the top sheet. Conventionally ADLs are made from loose long fibre (e.g. up to 100 mm) non-woven materials that use plastics such as polyester, polyethylene, polypropylene and other polyolefins as primary component(s). Conventionally ADLs are not biodegradable and, as they contain long fibres, are not easily dispersible and are not well suited for flushable sanitary pads.

To enable biodegradability and flushability, an ADL formed with a mixture of polyvinyl alcohol (PVA) fibres is provided. Other materials may be included such as wood pulp or viscose. The fibres may be similarly dimensioned and arranged as in a conventional ADL, laid in parallel and not dense. In an example the fibres have an average length in the range of 1-100 mm. In an example the fibres have an average diameter in the range of 1-100 μm. Alternative PVA fibre arrangements and shapes are also possible. The PVA fibres may be hollow tubes for instance. The PVA fibres can be formed by a conventional method, for example by an extrusion process such as spinning. The PVA fibres can disperse and dissolve in an excess of water. An ADL formed of or including a substantial component part of PVA fibres can create a material that aids in fluid distribution within a biodegradable and flushable sanitary pad, and can thus allow the performance of a sanitary pad to be increased.

To test water-dispersibility and water-solubility of the sanitary pad, the “Fine to Flush: Specification for a testing methodology to determine whether a product is suitable for disposal through a drain or sewer system” (WIS 4-02-06, November 2019 Issue 1.2) Disintegration in the drainline test is performed. Key steps of the Fine to Flush protocol include:
Pour 1 litre of tap water into a baffled shake flask and add the sample.
Agitate the flask on an orbital shaker for 3 hours at 100 rpm.
Pour the contents of the flask onto a 12.5 mm sieve submerged in a bath, taking care to spread the contents evenly over the plate.
agitate the submerged sieve backwards and forwards three times in the bath and lift out of the bath, rotate the sieve 45°, and repeat the submerge-agitate-lift sequence for 1 minute.
Collect the material retained on the upper surface and underside of the sieve and determine the proportion of material (by dry weight) passed through the sieve.

The sample is considered to disintegrate sufficiently if at least 50% of the sample mass passes through the 12.5 mm perforated plate sieve.

Alternatively the INDA EDANA “Guidelines for Assessing the Flushability of Disposable Nonwoven Products” (Edition 4, May 2018, Association of the Nonwoven Fabrics Industry (INDA); European Disposables and Nonwovens Association (EDANA)) Slosh Box Disintegration Test (4.2 FG502.R1(18)) is performed. Key steps include:
expose the sample to agitation in a box oscillating at 26 rpm with 2 litres of tap water for 60 minutes at 20° C.+/−3° C.;
pour the contents of the box onto a 12.5 mm perforated plate sieve resting at least 2 inches above a surface;
rinse for 120 seconds at 4 litres per minute flow rate;
collect the material retained on the sieve and determine the proportion of material (by dry weight) passed through the sieve.

The sample is considered to disintegrate sufficiently if more than 60% of the sample mass passes through the sieve for at least 80% of the individual replicates tested.

Alternatively the IWSFG “Publicly Available Specification (PAS) 3: 2020 Disintegration Test Methods—Slosh Box” (December 2020, International Water Services Flushability Group) Slosh Box Disintegration Test is performed. Key steps include:
expose the sample to agitation in a box oscillating at 18 rpm with 4 litres of tap water for 30 minutes at 15° C.+/−1° C.;
pour the contents of the box onto a 25 mm perforated plate sieve;
rinse for 60 seconds at 4 litres per minute flow rate;
collect the material retained on the sieve and determine the proportion of material (by dry weight) passed through the sieve.

The sample is considered to disintegrate sufficiently if more than 80% of the sample mass passes through the sieve.

To test biodegradability of the sanitary pad, the “Fine to Flush: Specification for a testing methodology to determine whether a product is suitable for disposal through a drain or sewer system” (WIS 4-02-06, April 2019 Issue 1.1) Appendix G ‘Determination of synthetic and non-synthetic organic components test’ is performed. The test is designed to dissolve natural organic material in a sample and leave synthetic organic material as a residue. The chemical dissolution provides a surrogate test to give information on the amount of natural biomass material (which can be assumed to be biodegradable) versus synthetic organic material (which may not be biodegradable). The test is performed as follows: a test sample is placed in a beaker in a fume hood and covered with 1000 ml of 14-15% sodium hypochlorite (NaClO) aqueous solution (bleach), then left for 24 hours, stirring at the beginning and end of the test period. The contents of the beaker are emptied onto a 0.5 mm sieve, and the sample is rinsed by pouring 2 litres of tap water from a 2 litre beaker over the sieve. The sample is considered biodegradable if no fibres or other visible residues remain on the sieve in 5 tests.

Alternatively the INDA EDANA “Guidelines for Assessing the Flushability of disposable Nonwoven Products” (Edition 4, May 2018, Association of the Nonwoven Fabrics Industry (INDA); European Disposables and Nonwovens Association (EDANA)) Aerobic Biodisintegration/Biodegradation Tests are performed. The Aerobic Biodisintegration Test measures the total mass of a sample retained on a 1 mm sieve after being incubated with activated sludge for 14 days at ambient laboratory temperature. The average dry mass percent of material passing the sieve should exceed 95%. The Biodegradation Test follows the OECD 301B method (as adopted by the OECD on 17 Jul. 1992, Organisation for Economic Cooperation and Development) providing a standardised biodegradation test that measures the evolution of carbon dioxide resulting from biodegradation.

Alternatively one of the six OECD 301A-F test methods (as adopted by the OECD on 17 Jul. 1992, Organisation for Economic Cooperation and Development) are performed to determine biodegradability in an aerobic medium.

The above examples are to be understood as illustrative examples, and modifications of detail can be made within the scope of the invention. For example, sanitary pads may be provided in a wide variety of shape and sizes and intended uses, with or without tabs. While the present invention has been described in the context of a sanitary pad, the arrangement may be adapted for other water-dispersible fluid absorbers, such as a wound dressing, an incontinence product, a diaper, a cleaning pad, or other uses where blood or other bodily fluids are taken up in a fluid absorber. Further examples are envisaged. It is to be understood that any feature described in relation to any one example may be used alone, or in combination with other features described, and may also be used in combination with one or more features of any other of the examples, or any combination of any other of the examples. Furthermore, equivalents and modifications not described above may also be employed without departing from the scope of the invention, which is defined in the accompanying claims.

Reference numerals appearing in the claims are by way of illustration only and shall have no limiting effect on the scope of the claims. The term ‘comprising’ as used herein preferably means ‘including’.

1. A method of attaching a release liner to a sanitary pad comprising:
applying a pattern of adhesive, preferably water based pressure sensitive adhesive, to a sheet of release liner material;
drying a sheet of release liner material comprising an adhesive pattern;
overlaying a sanitary pad on the dried release liner material and adhesive pattern; and
compressing the overlaid sanitary pad and dried release liner together so as to adhere the dried adhesive to the sanitary pad.

2. The method according to claim 1, wherein the adhesive is applied to the release liner material in the form of a spray or in the form of a direct coating using an aperture.

3. The method according to claim 1, wherein the adhesive is applied to the release liner material by steps comprising:
block coating a layer of adhesive onto a surface of the release liner;
applying to an exposed surface of the block coating a protective sheet, wherein the protective sheet comprises adherent portions and non-adherent portions arranged in a pattern corresponding to a desired adhesive pattern; and
removing the protective sheet, wherein on removal of the protective sheet areas of the block coating in contact with the adherent portions are removed from the release liner, and areas of the block coating in contact with the non-adherent portions are left on the release liner.

4. The method according to claim 3 wherein the non-adherent portions are siliconized portions, and the adherent portions are non-siliconized portions.

5. The method according to claim 4 further comprising manufacturing the protective sheet by patterning, preferably by gravure coating, a silicone onto a non-siliconized sheet to form the siliconized portions.

6. The method according to claim 1, wherein the adhesive is applied to the release liner material by steps comprising:
doctor blade coating and/or reverse roll coating a water-based adhesive onto a non-water-dispersible liner;
drying the adhesive; and
transferring the dried adhesive to the release liner.

7. The method according to claim 1, wherein the adhesive is applied to the release liner material by steps comprising:
patterning the release liner to comprise adherent portions and non-adherent portions arranged in a pattern corresponding to a desired adhesive pattern; and
block coating a layer of adhesive onto a surface of the release liner;
such that in use, on removal of the release liner areas of the block coating in contact with the non-adherent portions are removed from the release liner, and areas of the block coating in contact with the adherent portions are left on the release liner.

8. The method according to any one of claims 1 to 7 wherein the adhesive is a water-soluble or water-dispersible adhesive and/or wherein the release liner is a water-soluble or water-dispersible release liner.

9. A method of patterning an adhesive onto a release liner, wherein the method comprises:
block coating a layer of adhesive onto a surface of the release liner;
applying to an exposed surface of the block coating a protective sheet, wherein the protective sheet comprises adherent portions and non-adherent portions arranged in a pattern corresponding to a desired adhesive pattern; and
removing the protective sheet, wherein on removal of the protective sheet areas of the block coating in contact with the adherent portions are removed from the release liner, and areas of the block coating in contact with the non-adherent portions are left on the release liner.

10. The method according to claim 9 wherein the adhesive is a water-soluble or water-dispersible adhesive and/or wherein the release liner is a water-soluble or water-dispersible release liner.

11. The method according to claim 9 or 10 wherein the non-adherent portions are siliconized portions, and the adherent portions are non-siliconized portions.

12. The method according to claim 11, further comprising manufacturing the protective sheet by patterning, preferably by gravure coating, a silicone onto a non-siliconized sheet to form the siliconized portions.

13. A method of patterning a water-dispersible release liner, wherein the method comprises:
doctor blade coating and/or reverse roll coating a water-based adhesive onto a non-water-dispersible liner;
drying the adhesive; and
transferring the dried adhesive to the release liner.

14. The method according to claim 13 wherein transferring the adhesive to the release liner comprises contacting the patterned adhesive to the release liner.

15. The method according to claim 13 or 14 wherein the doctor blade (or a profile of a metering roll of the reverse roll coating) forms a number of gaps to coat the water-based adhesive onto the non-water-dispersible liner in a strip pattern.

16. The method according to any one of claims 13 to 15 wherein the water-based adhesive is a water-soluble or water-dispersible adhesive.

17. The method according to any one of claims 13 to 16 wherein the non-water-dispersible liner is a clay paper or a glassine paper.

18. A method of patterning a release liner, wherein the method comprises:
patterning the release liner to comprise adherent portions and non-adherent portions arranged in a pattern corresponding to a desired adhesive pattern; and
block coating a layer of adhesive onto a surface of the release liner;
such that in use, on removal of the release liner areas of the block coating in contact with the non-adherent portions are removed from the release liner, and areas of the block coating in contact with the adherent portions are left on the release liner.

19. The method according to claim 18 wherein the adhesive is a water-soluble or water-dispersible adhesive and/or wherein the release liner is a water-soluble or water-dispersible release liner.

20. The method according to claim 18 or 19 wherein the non-adherent portions are siliconized portions, and the adherent portions are non-siliconized portions.

21. The method according to claim 20, comprising patterning the release liner by patterning a silicone onto a non-siliconized sheet to form the siliconized portions, preferably by spraying or gravure coating.

22. A method of bonding a fibrous material and a polyvinyl alcohol film, comprising
applying a polyvinyl alcohol solution to a surface of the polyvinyl alcohol film and/or a surface of the fibrous material; and
contacting the surface of the polyvinyl alcohol with the surface of the fibrous material such that the applied polyvinyl alcohol solution is positioned between the polyvinyl alcohol film and the fibrous material.

23. The method according to claim 22 further comprising compressing the contacted polyvinyl alcohol film and fibrous material together, preferably in the absence of heating.

24. The method of claim 22 or 23 wherein the polyvinyl alcohol solution is one or more of:
a solution of high hydrolysis polyvinyl alcohol, preferably over 80 mol % hydrolysis;
1-15% by weight polyvinyl alcohol in solvent, preferably around 5% by weight polyvinyl alcohol in solvent;
an aqueous solution; and
applied at less than 5 gsm, preferably around 1 gsm.

25. The method according to claim 24, wherein the fibrous material and the polyvinyl alcohol film at least partially form a sublayer of a sanitary pad; preferably wherein the fibrous material and the polyvinyl alcohol film at least partially form a bottom sheet of a sanitary pad.

26. The method according to claim 22 or 23 wherein the polyvinyl alcohol solution is one or more of:
a solution of high hydrolysis polyvinyl alcohol, preferably over 80 mol % hydrolysis;
10-20% by weight polyvinyl alcohol in solvent, preferably around 15% by weight polyvinyl alcohol in solvent;
an aqueous solution; and
applied to form a film of more than 1 gsm (dry), preferably more than 3 gsm (dry); and/or
less than 15 gsm (dry), preferably less than 12 gsm (dry).

27. The method according to claim 26, wherein the fibrous material at least partially forms an absorbent core of a sanitary pad, and the polyvinyl alcohol film at least partially forms a bottom sheet of the sanitary pad.

28. The method according to any one of claims 22 to 27, wherein the fibrous material is a non-woven fabric or a loose fibre material.

29. A method of manufacturing a bottom layer of a sanitary pad, the method comprising:
priming a non-woven cellulosic fabric for application of an adhesive by applying an aqueous solution of polyvinyl alcohol or carboxymethylcellulose; and
drying the primed non-woven cellulosic fabric.

30. The method according to claim 29 wherein the aqueous solution is about 0.1-5% by weight polyvinyl alcohol or carboxymethylcellulose in water, preferably around 2% by weight polyvinyl alcohol in water.

31. The method according to claim 29 or 30 wherein the aqueous solution comprises a high hydrolysis polyvinyl alcohol, preferably with a degree of hydrolysis of over 80 mol %.

32. The method according to any one of claims 29 to 31 wherein the aqueous solution of polyvinyl alcohol or carboxymethylcellulose is applied by spraying, gravure or slot die.

33. The method according to any one of claims 29 to 32 wherein the primed non-woven cellulosic fabric comprises a coating of polyvinyl alcohol or carboxymethylcellulose that is less than 1 μm thick and/or less than 1 gsm.

34. The method according to any one of claims 29 to 33 further comprising contacting a pressure sensitive adhesive to a surface of the primed non-woven cellulosic fabric.

35. The method according to any one of claims 29 to 34 further comprising extruding or laminating or otherwise bonding a layer of polyvinyl alcohol onto an opposite surface of the non-woven cellulosic fabric.

36. A method of assembling a sanitary pad and release liner comprising:
overlaying a sanitary pad onto an adhesive pattern arranged on a release liner, wherein the sanitary pad is overlaid in a folded configuration in which tabs of the sanitary pad are folded over onto a main body of the sanitary pad.

37. The method according to claim 36 comprising:
cutting from the release liner a shape complementary to the shape of the sanitary pad in the unfolded configuration, wherein the cut release liner shape comprises wing sections extending from a central section of the cut release liner shape, with an adhesive pattern arranged on the wing sections;
folding the wing sections of the cut release liner onto the folded tabs of the sanitary pad;
and compressing the folded release liner so as to adhere the adhesive pattern and release liner to the tabs of the sanitary pad.

38. The method according to claim 37 comprising:
folding a first section of the release liner and sanitary pad to overlay the wing sections; and
folding a second section of the release liner and sanitary pad to overlay the first section.

39. A method of manufacturing a sanitary pad comprising an absorbent core, the method of manufacturing comprising:
perforating a shape into a sheet of absorbent core material;
overlaying the sheet of absorbent core with a top sheet;
embossing a shape and/or pattern into the overlaid top sheet and sheet of absorbent core,
wherein the embossed shape and/or pattern secures the top sheet to the absorbent core; and
removing excess absorbent core material from around the perforated shape to leave a top sheet and an absorbent core, wherein the absorbent core is substantially the shape of the perforated shape.

40. The method according to claim 39 further comprising:
overlaying the top sheet and absorbent core on a bottom sheet; and
sealing the top sheet and the bottom sheet together.

41. The method according to claim 39 or 40 further comprising:
overlaying the sheet of absorbent core on a sublayer comprising a polyvinyl alcohol film; and
perforating the shape into both the sheet of absorbent core material and the sublayer.

42. The method according to any one of claims 39 to 41 wherein the absorbent core sheet, top sheet and bottom sheet are continuous webs of material.

43. The method according to any one of claims 39 to 42 wherein 90 to 99% of the perimeter of the perforated shape comprises perforation holes, preferably wherein 95% of the perimeter of the shape comprises perforation holes.

44. The method according to any one of claims 39 to 43 wherein the embossed pattern comprises at least one fluid distribution channel extending within the perforated shape for distributing fluid within a sanitary pad.

45. The method according to claim 44 wherein the fluid distribution channels comprise: a plurality of longitudinal channels; and a number of side channels branching out from the longitudinal channels, preferably in connection with outer adjacent longitudinal channels.

46. The method according to claim 44 or 45 wherein the fluid distribution channels comprise: a central longitudinal channel wherein the width of the longitudinal channel varies.

47. A sanitary pad produced by the method of any one of claims 1-46.

48. An acquisition distribution layer for a sanitary pad comprising long polyvinyl alcohol fibres.

49. The acquisition distribution layer of claim 48 wherein the long polyvinyl alcohol fibres are at least 1 mm long, preferably at least 3 mm long, preferably at least 10 mm long, preferably at least 20 mm long, preferably at least 30 mm long.

50. The acquisition distribution layer of claim 48 or 49, wherein the polyvinyl fibres are hollow and tubular in shape.

51. The acquisition distribution layer of any one of claims 48 to 50 formed partially or substantially of polyvinyl alcohol fibres.

52. The acquisition distribution layer of any of claims 48 to 51 wherein the polyvinyl alcohol fibres are arranged in parallel to form a non-woven fabric.

53. A water dispersible sanitary pad comprising:
a top layer of a non-woven cellulosic fabric, preferably having around 60 gsm weight;
an absorbent layer comprising:
fiberised cellulose wood pulp, preferably having around 250 gsm weight;
a super absorbent polymer, preferably a cellulose gum, further preferably around 1 g of super absorbent polymer per sanitary pad; and
optionally a first carrier layer of a non-woven cellulosic fabric, preferably having around 60 gsm weight;
a sublayer comprising:
a polyvinyl alcohol film, preferably around 20 μm or less thick; and
optionally a second carrier layer of a non-woven cellulosic fabric, preferably having around 60 gsm weight;
a bottom sheet comprising:
a polyvinyl alcohol film, preferably around 30 μm thick; and
a layer of a non-woven cellulosic fabric, preferably having around 60 gsm weight;
optionally a polyvinyl alcohol film, preferably less than 1 μm thick; and
a water-soluble pressure sensitive adhesive, preferably included at around 8-10 gsm.

54. The sanitary pad of claim 53 further comprising a release liner comprising:
a silicone layer, preferably having around 1-4 gsm;
a polyvinyl alcohol film, preferably at least 10 μm thick; and
a layer of a non-woven cellulosic fabric, preferably having around 60 gsm weight.

55. A sanitary pad comprising:
an absorbent core;
a bottom layer comprising a non-woven fabric and a polyvinyl alcohol film; and
a sublayer arranged between the absorbent core and the bottom layer, the sublayer comprising a polyvinyl alcohol film and optionally a non-woven fabric.

56. The sanitary pad of claim 55 wherein the polyvinyl alcohol film of the sublayer is provided adjacent to the absorbent core; preferably wherein the absorbent core comprises a non-woven fabric and the polyvinyl alcohol film of the sublayer is provided adjacent to the non-woven fabric of the absorbent core.

57. The sanitary pad of claim 55 or 56 wherein the polyvinyl alcohol film of the sublayer is between 10 and 30 μm thick, preferably between 15 and 25 μm thick, and further preferably around 20 μm thick.

58. The sanitary pad of any one of claims 55 to 57 wherein the non-woven fabric of the sublayer and/or the non-woven fabric of the bottom layer is formed of a non-woven wet laid cellulosic fibre material, preferably having around 60 gsm weight.

59. The sanitary pad of any of claims 55 to 58 wherein the polyvinyl alcohol film of the bottom layer is between 10 and 80 μm thick, preferably between 30 and 50 μm thick, and further preferably around 30 μm thick.

60. The sanitary pad of any of claims 55 to 59 comprising two or more sublayers arranged between the absorbent core and the bottom layer.

61. The sanitary pad of any of claims 55 to 60 wherein the sublayer is a bonding sublayer formed by application of a polyvinyl alcohol solution to the polyvinyl alcohol film of the bottom layer.

62. The sanitary pad of claim 61 wherein the polyvinyl alcohol film of the sublayer has a dry weight of more than 1 gsm, preferably more than 3 gsm; and/or less than 15 gsm, preferably less than 12 gsm.

63. The sanitary pad of claim 61 or 62 wherein the polyvinyl alcohol solution is in the range of 10-20% by weight polyvinyl alcohol in water, preferably around 15%.

64. The sanitary pad of any one of claims 55 to 63 wherein the polyvinyl alcohol solution is a solution of high hydrolysis polyvinyl alcohol, preferably over 80 mol % hydrolysis.
